(19) United States
(12) Patent Application Publication
(10) Pub. No.: US 2022/0301699 A1
(43) Pub. Date: Sep. 22, 2022

(54) SYSTEM AND METHOD FOR A CONTINUOUS PATIENT ENGAGEMENT ORAL CARE MODEL

(71) Applicant: NOHO DENTAL, INC. d/b/a TEND, Nashville, TN (US)

(72) Inventors: Mark Edwin Crockett, Waverly, TN (US); Benjamin Grohe, Brooklyn, NY (US)

(73) Assignee: NOHO DENTAL, INC. d/b/a TEND, Nashville, TN (US)

(21) Appl. No.: 17/697,343

(22) Filed: Mar. 17, 2022

Related U.S. Application Data
(60) Provisional application No. 63/162,381, filed on Mar. 17, 2021.

(51) Int. Cl. G16H 40/20; G06Q 10/10

(52) U.S. Cl. CPC G16H 40/20; G06Q 10/1095

(57) ABSTRACT

A continuous patient engagement oral care model configured to boost patient trust and loyalty which will lead to stronger relationships between patients and oral care providers and more consistent visits from the patients. A Cloud-based system and method implemented for such a continuous patient engagement oral care model may be configured to identify treatments that are important to particular patients, propose treatment for consideration or investigation, and solicit feedback on relevance and priority of the proposed treatment plans.

### CROSS REFERENCE TO RELATED APPLICATION

This application claims priority to U.S. Provisional Patent Application No. 63/162,381 filed on Mar. 17, 2021, entitled “DIGITAL WORKFLOW SYSTEMS AND METHODS,” the content of which is incorporated by reference herein in its entirety.

### FIELD OF TECHNOLOGY

The present disclosure generally relates to a system and method of a data-driven clinical model, and more particularly relates to a system and method for a continuous patient engagement oral care model.

### BACKGROUND

Oral health is closely related to one's physical health, medical costs and quality of life. Traditionally, when visiting a dentist, patients may not provide information in advance of their visit and often are providing it in the waiting room or in the dentist's chair. This includes estimating the cost of the visit and validating insurance coverage. This may delay the start of the visit and fail to tailor patients' experience to their personal preferences and clinical needs.

Patient engagement has been considered to be a key element of high quality evidence-based clinical practice. However, a patient's fear and anxiety toward dentists and dental treatments are both significant characteristics that contribute to avoidance of dental care. Whether they let a toothache linger for too long or feel embarrassed about their teeth or breath, some people fear being judged or shamed by their dentists, or getting bad news. Anxiety associated with the thought of visiting the dentist for preventive care and over dental procedures is referred to as dental anxiety. Traditional oral health practices have been struggling to address this frequently encountered problem in dental offices.

Accordingly, there is a need for a continuous patient engagement oral care model that is configured to boost patient trust and loyalty which will lead to stronger relationships between patients and oral care providers and more consistent visits from the patients. With continuous patient engagement, healthcare providers can identify treatments that are important to particular patients, propose treatment for consideration or investigation, and solicit feedback on relevance and priority of the proposed treatment plans. Moreover, healthcare providers and patients can have meaningful education communications on an ongoing basis that highlight the benefits of healthy teeth and gums and the consequences of neglect, thereby helping the patients keep up with recommended care at home or with in-office treatments.

### SUMMARY

The present disclosure generally relates to a system and method configured to provide a continuous patient engagement oral care model. Among other features, the present disclosure relates to a system deployed within a Cloud-based communication network. The system may comprise a mobile device, comprising: a non-transitory computer-readable storage medium configured to store an application program; and a processor coupled to the non-transitory computer-readable storage medium and configured to control a plurality of modules to execute instructions of the application program for generating a request for an appointment at a provider location, wherein the request comprises data representing a phone number, an email address, a service type of the appointment, the provider location, and a service date and time.

The system may also comprise a first computing server system configured to: receive the request, determine whether the request is associated with a new patient or an existing patient of the system, generate boundary conditions based at least upon the service date for inquiring information saved in a second computing server system relating to active appointments of the provider location, blocked availability and operation hours of the provider location, generate a set of time spans based on the information obtained from the second computing server system to determine unavailability at the provider location, invert each of the set of time spans to obtain available time spans at the provider location, determine a service duration based on the service type of the appointment and whether the request is associated with the new patient or the existing patient of the system. In response to detecting that the request is associated with the new patient, the first computing server system may assign a first of the available time spans based on the service duration. In response to detecting that the request is associated with the existing patient, the first computing server system may assign a second of the available time spans based on the service duration. Further, a first computing server system may transmit the first or the second of the available time spans to the application program of the mobile device. In response to detecting a confirmation of either the first or the second of the available time spans via the application program of the mobile device, the first computing server system may validate booking information of the appointment and transmit the booking information of the appointment to the second computing server system.

In one embodiment, the first computing server system may be configured to determine whether the request is associated with the new patient or the existing patient of the system by at least checking the phone number and the email address against records saved in a database associated with the first computing server system. The first computing server system may create a patient record in response to detecting that the phone number and the email address have no match in the records saved in the database associated with the first computing server system, wherein the patient record comprises a chart number uniquely assigned to the new patient. Further, the first computing server system may be configured to assign a first identifier to each member of the system and the second computing server system may be configured to assign a second identifier to each patient, and the first and second computing server systems may be configured to synchronize patient records in accordance with a selected time interval using the chart number, the first identifier, and the second identifier.

In another embodiment, the first computing server system may be configured to retrieve patient data from the second computing server system and present at least a portion of the patient data via the application program of the mobile device in response to determining that the request is associated with the existing patient of the system. In one aspect, the first computing server system may be configured to concurrently receive another request from another mobile device for the appointment at the provider location, determine which request has been confirmed first, and generate a message to redirect a later-confirmed request to another booking process.

The first computing server system may be further configured to determine any of the available time spans is at least equal to the service duration, synchronize at least a portion of the booking information with a local calendar and map application program of the mobile device, and generate and transmit one or more notifications to the application program of the mobile device prior to the appointment. The one or more notifications may prompt inputs of at least insurance coverage information, health information, and financial information from a patient. The first computing server system may further be configured to reschedule the appointment for the patient if the insurance coverage information, health information, or financial information is not received within a selected period of time prior to the appointment.

Among other features, the present disclosure relates to a computer-implemented method. The method may comprise generating, by a processor of a mobile device deployed within a Cloud-based communication network, a request for an appointment at a provider location, wherein the request comprises data representing a phone number, an email address, a service type of the appointment, the provider location, and a service date and time; receiving, by a first computing server system deployed within the Cloud-based communication network, the request; determining, by the first computing server system, whether the request is associated with a new patient or an existing patient of an oral care system; generating, by the first computing server system, boundary conditions based at least upon the service date for inquiring information saved in a second computing server system relating to active appointments of the provider location, blocked availability and operation hours of the provider location; generating, by the first computing server system, a set of time spans based on the information obtained from the second computing server system to determine unavailability at the provider location; inverting, by the first computing server system, each of the set of time spans to obtain available time spans at the provider location; determining, by the first computing server system, a service duration based on the service type of the appointment; in response to detecting that the request is associated with the new patient, assigning, by the first computing server system, a first of the available time spans based on the service duration; in response to detecting that the request is associated with the existing patient, assigning, by the first computing server system, a second of the available time spans based on the service duration; transmitting, by the first computing server system, the first or the second of the available time spans to the application program of the mobile device; and in response to detecting a confirmation of either the first or the second of the available time spans via the application program of the mobile device, validating, by the first computing server system, booking information of the appointment and transmitting the booking information of the appointment to the second computing server system.

The method may further comprise determining, by the first computing server system, whether the request is associated with the new patient or the existing patient by at least checking the phone number and the email address against records saved in a database associated with the first computing server system, and creating, by the first computing server system, a patient record in response to detecting that the phone number and the email address have no match in the records saved in the database associated with the first computing server system, wherein the patient record comprises a chart number uniquely assigned to the new patient.

In one embodiment, the method may comprise assigning, by the first computing server system, a first identifier to each member of the system; assigning, by the second computing server system, a second identifier to each patient; and synchronizing patient records saved in the first and second computing server systems in accordance with a selected time interval using the chart number, the first identifier, and the second identifier.

In one example, the method may comprise concurrently receiving, by the first computing server system, another request from another mobile device for the appointment at the provider location; determining which request has been confirmed first; and generating a message to redirect a later-confirmed request to another booking process.

The present disclosure may also relate to a non-transitory computer readable medium storing computer executable instructions for a system deployed in a Cloud-based communication network, the instructions being configured for generating, by a processor of a mobile device deployed within the Cloud-based communication network, a request for an appointment at a provider location, wherein the request comprises data representing a service type of the appointment, the provider location, and a service date and time; receiving, by a first computing server system deployed within the Cloud-based communication network, the request; determining, by the first computing server system, whether the request is associated with a new patient or an existing patient of an oral care system; generating, by the first computing server system, boundary conditions based at least upon the service date for inquiring information saved in a second computing server system relating to active appointments of the provider location, blocked availability and operation hours of the provider location; generating, by the first computing server system, a set of time spans based on the information obtained from the second computing server system to determine unavailability at the provider location; inverting, by the first computing server system, each of the set of time spans to obtain available time spans at the provider location; determining, by the first computing server system, a service duration based on the service type of the appointment; in response to detecting that the request is associated with the new patient, assigning, by the first computing server system, a first of the available time spans based on the service duration; in response to detecting that the request is associated with the existing patient, assigning, by the first computing server system, a second of the available time spans based on the service duration; transmitting, by the first computing server system, the first or the second of the available time spans to the application program of the mobile device; and in response to detecting a confirmation of either the first or the second of the available time spans via the application program of the mobile device, validating, by the first computing server system, booking information of the appointment and transmitting the booking information of the appointment to the second computing server system.

The non-transitory computer readable medium of the present disclosure may also comprise instructions for determining, by the first computing server system, whether the request is associated with the new patient or the existing patient by at least checking the phone number and the email address against records saved in a database associated with the first computing server system.

Additionally, the non-transitory computer readable medium of the present disclosure may comprise instructions for creating, by the first computing server system, a patient record in response to detecting that the phone number and the email address have no match in the records saved in the database associated with the first computing server system, wherein the patient record comprises a chart number uniquely assigned to the new patient.

Moreover, the non-transitory computer readable medium may further comprise instructions for: assigning, by the first computing server system, a first identifier to each member of the system; assigning, by the second computing server system, a second identifier to each patient; and synchronizing patient records saved in the first and second computing server systems in accordance with a selected time interval using the chart number, the first identifier, and the second identifier.

The above-simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplary pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 illustrates a system for a continuous patient engagement oral care model, according to aspects of the present disclosure;

FIG. 2 illustrates a Cloud-based management server system, according to aspects of the present disclosure;

FIGS. 3(a)-3(h) illustrate example screenshots when an onboarding member of the system 100 of FIG. 1 makes an appointment with a dental care provider via a mobile or web-based application, according to aspects of the present disclosure;

FIGS. 4(a)-4(e) illustrate example screenshots for obtaining insurance information when an onboarding member of the system 100 of FIG. 1 makes an appointment with a dental care provider via a mobile or web-based application, according to aspects of the present disclosure;

FIGS. 5(a)-5(g) illustrate example screenshots for personalizing an upcoming visit when an onboarding member of the system 100 of FIG. 1 makes an appointment with a dental care provider via a mobile or web-based application, according to aspects of the present disclosure;

FIG. 6 illustrates an example screenshot for obtaining payment information when an onboarding member of the system 100 of FIG. 1 makes an appointment with a dental care provider via a mobile or web-based application, according to aspects of the present disclosure;

FIGS. 7(a)-7(h) illustrate example screenshots for personalizing an upcoming visit when an onboarding member of the system 100 of FIG. 1 makes an appointment with a dental care provider via a mobile or web-based application, according to aspects of the present disclosure;

FIG. 8 illustrates a process of automated messaging workflows based on obtained member data relating to an upcoming visit, according to aspects of the present disclosure;

FIGS. 9(a)-9(e) illustrate example screenshots of a variety of notifications to a member who has completed a visit, according to aspects of the present disclosure;

FIGS. 10(a)-10(r) illustrate example screenshots of a mobile or web-based application, according to aspects of the present disclosure;

FIG. 11 illustrates a flow chart of an insurance verification process, according to aspects of the present disclosure; and

FIG. 12 illustrates a flow chart of an appointment booking process, according to aspects of the present disclosure.

### DETAILED DESCRIPTION

Various aspects of the present disclosure will be described with reference to the drawings, wherein like reference numerals are used to refer to like elements throughout. In the following description, for purposes of explanation, numerous specific details are set forth in order to promote a thorough understanding of one or more aspects of the present application. It may be evident in some or all instances, however, that any aspects described below can be practiced without adopting the specific design details described below.

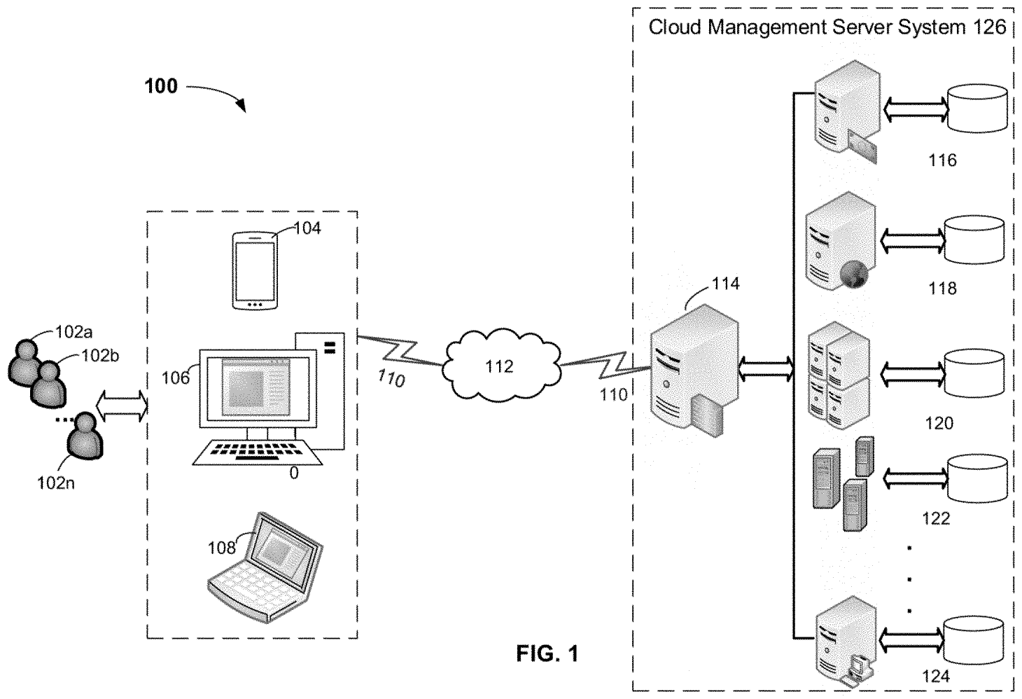

Referring to FIG. 1, in accordance with aspects of the present disclosure, a system 100 deployed within a Cloud-based computing environment and communication network may be configured to provide a seamless digital-to-physical member experience that captures information from dental practice onboarding members (e.g., at least one of users 102a, 102b . . . 102n) prior to their visits. For example, when a member schedules an appointment via a mobile or web-based application (e.g., native iOS or Android apps) downloaded and installed on a selected computing device 104, 106 or 108 and visits one of the studios associated with the system 100, a backend remote Cloud management server system 126 of the system 100 may be configured to provide an experience that is curated to deliver a bespoke experience that is based on the services that interest the patient. The term “server” generally refers to a computing device or system, including processing hardware and process space(s), an associated storage medium such as a memory device or database, and, in some instances, a database application as is well known in the art. It should also be understood that “server system” and “server” are often used interchangeably herein.

In one aspect, the Cloud management server system 126 may implement a management computing device/interface 114 configured to process, transform and exchange various information obtained from disparate data sources 114, 116, 118, 120, 122, 124 (3^{rd} party and/or proprietary to the system 100), including bi-directional data exchange and automation of task workflows, to create a seamless member experience. It should be appreciated that data storage capacity, processing power and networking of the Cloud management server system 126 may all be scaled (e.g., increasing or decreasing various information technology (IT) resources as needed to meet changing demand) using the Cloud computing infrastructure disclosed in accordance with the present disclosure. In one embodiment, the Cloud management server system 126 may be scaled vertically by adding or subtracting power to an existing Cloud server by upgrading random access memory (RAM), storage or processing power (e.g., central processing unit (CPU)). This type of scaling may have an upper limit based on the capacity of a specific server or machine being scaled and scaling beyond that may require downtime. Further, the Cloud management server system 126 may be scaled horizontally by adding more servers (e.g., servers) to distribute workload across machines, which in turn increases performance and storage capacity. This type of scaling may require minimal downtime. In addition, the Cloud management server system 126 may be configured to grow or shrink dynamically in response to changing workload demands, such as a sudden spike in Internet traffic. Such elasticity of the Cloud management server system 126 may automatically adapt to match resources with demand as closely as possible in real time. For example, when the system 100 experiences variable and unpredictable workloads (e.g., a sudden or seasonal demand at one or more studios of the system 100 due to a popular product introduction or social media boost), the Cloud management server system 126 may be configured to scale virtual desktop infrastructure in the Cloud environment for the system 100 for additional temporary studio staff, contractors, dental care providers/practitioners or for scheduling additional appointments such as tele-consults.

Among other features, the present disclosure relates to novel clinical treatment planning systems and processes. Here. “treatment planning” may generally refer to a recommended treatment by the general dentist following diagnosis. Traditionally, following a visit, if a dentist has recommended treatment, the patient may have very little context for what has been diagnosed, what the recommended treatment is, and how much it will cost based on the patient's insurance. In some embodiments, the present disclosure may be configured to offer payment plans, pre-approval, and flex payment options to help the patient consider her options. All services and functions of the present disclosure are provided in a Health Insurance Portability and Accountability Act of 1996 (HIPAA) compliant digital interface.

Computing devices 104, 106, 108 and any connected computing devices of the system 100 may be configured to communicate with the Cloud management server system 126 via a communication network 112 using suitable network connections and protocols 110. A communication network (e.g., communication network 112) may refer to a geographically distributed collection of computing devices or data points interconnected by communication links and segments for transporting signals and data therebetween. A protocol (e.g., protocol(s) 110) may refer to a set of rules defining how computing devices and networks may interact with each other, such as frame relay, internet protocol (IP), transmission control protocol (TCP), user datagram protocol (UDP), hypertext transfer protocol (HTTP). Many types of communication networks are available, ranging from local area networks (LANs), wide area networks (WANs), cellular networks, to overlay networks and software-defined networks (SDNs), a packet data network (e.g., the Internet), mobile telephone networks (e.g., cellular networks, such as 4G or 50), Plain Old Telephone (POTS) networks, and wireless data networks (e.g., Institute of Electrical and Electronics Engineers (IEEE) 802.11 family of standards known as Wi-Fi®, WiGig®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, virtual private networks (VPN), Bluetooth, Near Field Communication (NFC), or any other suitable network. Computing devices 104, 106 and 108 may be configured to communicate in a peer to peer manner to replace, duplicate, supplement or extend the functionalities of communication network 112.

For example, communication network 112 may be a LAN configured to connect each of computing devices 104, 106, 108 and other devices deployed within a full-service dental studio over dedicated private communications links. Communication network 112 may be a WAN configured to connect computing devices deployed within the full-service dental studio and other geographically dispersed computing devices and networks over long-distance communications links, such as common carrier telephone lines, optical light paths, synchronous optical networks (SONET), or synchronous digital hierarchy (SDH) links. The Internet may be used to connect disparate devices and networks throughout the world, providing global communication among nodes (a node of an Internet has an IP address) on various networks. These nodes may communicate over the communication network 112 by exchanging discrete frames or packets of data according to protocols 110, such as TCP/IP. Communication network 112 may be further interconnected by an intermediate network node, such as a router and/or gateway device, to extend the effective size of each network.

As described above, the Cloud management server system 126 of the present disclosure provides various computing services using shared resources. Cloud computing may generally include Internet-based computing in which computing resources are dynamically provisioned and allocated to each connected computing device or other devices on-demand, from a collection of resources available via the network or the Cloud. Cloud computing resources may include any type of resource, such as computing, storage, and networking. For instance, resources may include service devices (firewalls, deep packet inspectors, traffic monitors, load balancers, etc.), computing-processing devices (servers, CPUs, GPUs, random access memory, caches, etc.), and storage devices (e.g., network attached storages, storage area network devices, hard disk drives, solid-state devices, etc.). In addition, such resources may be used to support virtual networks, virtual machines, databases, applications, etc. The term “database,” as used herein, may refer to a database (e.g., relational database management system (RDBMS) or structured query language (SQL) database), or may refer to any other data structure, such as, for example a comma separated values (CSV), tab-separated values (TSV). JavaScript Object Notation (JSON), eXtendible markup language (XML), TeXT (TXT) file, flat file, spreadsheet file, and/or any other widely used or proprietary format. In some embodiments, one or more of the databases or data sources may be implemented using one of relational databases, flat file databases, entity-relationship databases, object-oriented databases, hierarchical databases, network databases, NoSQL databases, and/or record-based databases.

Within the system 100, Cloud computing resources accessible via any suitable communication network (e.g., Internet) may include a private Cloud, a public Cloud, and/or a hybrid Cloud. Here, a private Cloud may be a Cloud infrastructure operated by an enterprise for use by the enterprise, while a public Cloud may refer to a Cloud infrastructure that provides services and resources over a network for public use. In a hybrid Cloud computing environment which uses a mix of on-premises, private cloud and third-party, public Cloud services with orchestration between the two platforms, data and applications may move between private and public Clouds for greater flexibility and more deployment options. Some example public Cloud service providers may include Amazon (e.g., Amazon Web Services® (AWS)), IBM (e.g., IBM Cloud), Google (e.g., Google Cloud Platform), and Microsoft (e.g., Microsoft Azure®). These providers provide Cloud services using computing and storage infrastructures at their respective data centers and access thereto is generally available via the Internet. Some Cloud service providers (e.g., Amazon AWS Direct Connect and Microsoft Azure ExpressRoute) may offer direct connect services and such connections typically require users to purchase or lease a private connection to a peering point offered by these Cloud providers.

As shown in FIG. 1, the backend remote Cloud management server system 126 may comprise a management computing device/interface 114 configured to connect with a plurality of 3^{rd} party and/or proprietary software systems, computing platforms or systems 116, 118, 120, 122, and 124 to receive and process requests from various sources and deliver requested data and services. In accordance with aspects of the present disclosure, the management computing device/interface 114 may be configured to handle protocol translation, service discovery, basic business logic, authentication and security policy enforcements, stabilization and load balancing, cache management and various monitoring, logging and analytics functions. In one embodiment, the management computing device/interface 114 may be an external endpoint made available to consumers of various services provided by the system 100, and may encapsulate the business functionality of the overall architecture of the system 100. For example, the management computing device/interface 114 may include an application programming interface (API) gateway device which may function as a common entry point for some or all clients (desktop, mobile, tablet or hubs (e.g., member computing devices 104, 106 and 108 and computing devices associated with each dental studio and dental care provider of the system 100)) accessing various resources and services provided by the system 100.

It should be appreciated that each of the computing devices/systems 114, 116, 118, 120, 122, and 124 may comprise at least one of computing devices, servers, server farms, laptops, tablets, mobile devices, smart phones, smart watches, fitness tracker devices, cellular devices, gaining devices, media players, network enabled printers, routers, wireless access points, network appliances, storage systems, any suitable databases, gateway devices, smart home devices, virtual or augmented reality devices, or any other suitable devices that are deployed in the same or different communication networks of these computing devices and systems. The Cloud management server system 126 may be configured to provide functionalities for any connected devices such as sharing data or provisioning resources among multiple client devices, or performing computations for each connected client device.

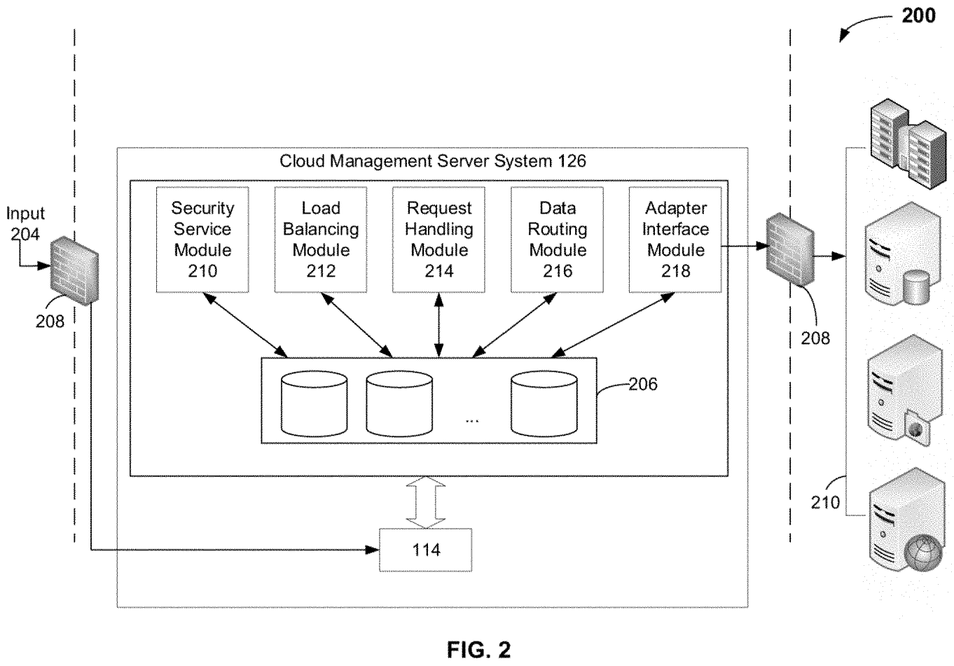

FIG. 2 shows an example architecture 200 of the system 100 of FIG. 1 using a Cloud management computing server system 126 for exchanging information among different entities, according to aspects of the present disclosure. On a high level, the Cloud management computing server system 126 may be configured to facilitate on-demand delivery of computing power, database storage, software applications, and other IT resources through the management computing device/interface 114 and the Internet (e.g., communication network 112). The Cloud management computing server system 126 may include multiple Cloud servers concurrently running on a hypervisor to control the capacity of underlying operating systems and allocate processor cycles, memory space, network bandwidth and so on. Input 204 (e.g., data and messages) to the Cloud computing server system 126 may be obtained from computing devices 104, 106, 108, or other data sources or computing devices connected therewith via the management computing device/interface 114. In one embodiment, input 204 may include Cloud service invocation messages, result messages, request messages, or any messages communicated among different Cloud computing devices. For example, a message may include a message type (e.g., a type value from a set of shared type constants), a unique identifier (e.g., an identifier used to correlate this message with one or more other messages), priority information to support priority based message queues, timeout, sensitivity indicator to support message data isolation, message source (e.g., a uniform resource identifier (URI) of a sender), a message destination (e.g., a URI that uniquely identifies the destination), a request context (e.g., request information from a dispatcher), and/or a message payload. The payload may have different attributes depending upon the type of message that is being sent, such as parameter data and result data.

The Cloud computing server system 126 may be configured to operate as a secure intermediary computing environment for real time or near real time data collection, storage, and analysis in connection with the use of e.g., devices 104, 106, 108. In one embodiment, the Cloud computing server system 126 and the management computing device/interface 114 may implement techniques to facilitate communications among various mobile computing devices and Cloud computing entities including but not limited to Cloud datacenters, Cloud web servers, Cloud application servers. Cloud database servers, Cloud storage devices, despite their incompatibilities in communication, such as differences between formats or communication protocols. In certain embodiments, the management computing device/interface 114 may be configured to translate communication protocols among different computing devices.

The Cloud computing server system 126 and the management computing device/interface 114 may be implemented using hardware, software, firmware, or combinations thereof. As shown in FIG. 2, the Cloud computing server system 126 may include one or more computing devices, such as a computing server device, one or more memory storage data repositories 206, one or more processors, and operate with different kinds of operating systems. Each memory storage device may implement one or more databases (e.g., a document database, a relational database, or other type of database), one or more file stores, one or more file systems, or combinations thereof, and may include instructions stored thereon which, when executed by the processor(s), cause the processor(s) to implement one or more operations disclosed herein.

Data repositories 206 may be accessible by various modules 210-218. The term “module,” “engine,” “interface,” “gateway,” “element,” or “component” as used herein refers to a real-world device, component, or arrangement of components implemented using hardware, such as by an application specific integrated circuit (ASIC) or field-programmable gate array (FPGA), for example, or as a combination of hardware and software, such as by a microprocessor system and a set of instructions to implement certain functionalities, which (while being executed) transform the microprocessor system into a special-purpose device. A “module,” “engine,” “interface.” “gateway,” “element,” or “component” can also be implemented as a combination of the two, with certain functions facilitated by hardware alone, and other functions facilitated by a combination of hardware and software.

For example, one of the data repositories 206 may store all the metadata (e.g., run-time and design-time data, each having their own requirements on availability and performance) associated with the server system 126. A connected computing device (e.g., devices 104, 106. 108) of the system 100 may have any number of applications installed thereon. Each application may be versioned and have at least one versioned resource application API, and corresponding versioned service. The data repository may store one or more callable interfaces, which may be invoked by device 104, 106, 108. The callable interface may be implemented to translate between one format, protocol, or architectural style for communication and another format, protocol, or architectural style for communication. Further, another data repository 206 may be used to store information about processing occurring in the system 100, such as messages communicated via the management computing device/interface 114 and log information. Additional data repositories 206 may be configured to store logging and analytics data captured during processing in the Cloud computing server system 126. Depending on the demand of computing devices seeking to communicate with backend Cloud resources 116, 118, 120, 122 and 124, the Cloud computing server system 126 may be configured to handle surges and temporary periods of higher than normal traffic between each mobile computing device and other Cloud computing devices. For example, the management computing device/interface 114 may include modules or elements that support scalability such that components may be added or replaced to satisfy demand in communication.

Input 204 (e.g., a request for a Cloud service) may be communicated between device 104, 106, or 108 and the management computing device/interface 114 via one or more callable interfaces, e.g., APIs. The Cloud computing server system 126 may be protected by one or more firewalls 208 to provide a secure environment to process requests from various computing devices. For example, firewalls 208 may permit communication of messages between the Cloud computing server system 126 and each device 104, 106, and/or 108. Such messages (e.g., SPDY messages, hypertext transfer protocol (HTTP) messages or representational state transfer (REST) messages) may conform to a communication protocol (e.g., SPDY, HTTP, or REST). Input 204 that is received through the firewall 208 may be processed by security service module 210 which is configured to manage security authentication for a user associated with a service request by at least restricting access to only those who have the required credentials to certain personal and/or professional data. In one embodiment, security authentication may be determined for a request, a session, a user, a device, other criterion related to the user, or combinations thereof. Security authentication may be performed for each request that is received or based on a previous verification of a request. Security authentication may be determined for a user or a device, such that requests to different Cloud services 210 (e.g., Cloud resources 116, 118, 120, 122 and 124 in FIG. 1) may be authenticated based on a single verification of security.

Upon determining security authentication, a load balancing module 212 may be configured to detect to which Cloud service 210 the received request is directed, and use a request handling module 214 to transmit each service request to an appropriate Cloud service 210. A request may be routed to an appropriate service 210 upon dispatch, or to another module of the Cloud management server system 126. The request handling module 214 may resolve a request to determine its destination based on a location (e.g., a URI of the request). The request handling module 214 may parse a request's header to extract one or more of the following information: tenant identifier, service identifier, application name, application version, request resource, operation and parameters, etc. The request handling module 214 may use the parsed information to perform a lookup in data repositories 206 and retrieve corresponding application metadata. The request handling module 214 may determine the target service based on the requested resource and the mappings in the stored metadata. Via formatting the request and any other necessary information, the request handling module 214 may transmit the input message to the data routing module 216 for further processing, or on a queue and await the corresponding response. The request handling module 214 may process responses received from the data routing module 216 and return a response to, e.g., at least one of the devices 104, 106, 108.

The data routing module 216 may manage delivery of messages to destinations registered with itself. The data routing module 216 may operate as a central system for managing communications in Cloud services 210, such that additional centralized services (additional authorization, debugging, etc.) may be plugged in as necessary. Data captured by the data routing module 216 may be stored in the data repositories 206.

The data routing module 216 may route messages to one or more Cloud resources 210 directly, or with the aid of an adapter interface module 218 by translating or converting a message to a protocol supported by a receiving Cloud resource 210. The adapter interface module 218 may establish separate communication connections with each of Cloud resources 210.

In accordance with aspects of the present disclosure, the Cloud management server system 126 may be configured to obtain real time data from devices 104, 106, 108, and/or other data sources, such as stored historical data, conduct data capture, storage, analysis, search, share, transfer, query, and update of the obtained data using proprietary algorithms, and provide feedback to a user (e.g., users 102a, 102b . . . 102n of FIG. 1) in a real time, near real time, daily, monthly, or at a user requested interval.

In one embodiment, the management computing device/interface 114 may be configured to manage a web application which is a public facing website for handling authentication using e.g., OpenID Connect with AD domain services, provide data for marketing pages, and provide interfaces for all enterprise and system management. For example, an enterprise management interface may be provided to add-remove internal or external users from an enterprise subscription, and assign roles to internal users. Each subscription may be assigned a defined number of seats. Users with permission to invite other users may be given a certain number of seats they can invite others to use. Such enterprise management interface may be configured to transfer control of system 100 from one administrative user to another and display shared contact information for internal or external users. Moreover, a system management interface may be provided to allow administrators of system 100 to assist users with common tasks including but not limited to changing emails associated with user accounts if users are unable to use the self-service option in the application, changing passwords if users are unable to reset the passwords themselves, and displaying subscription information for each user. The system management interface may also be configured to manage enterprise subscriptions via options including creating, modifying, and deleting certain subscription information, and inviting administrators to enterprise subscriptions.

Various data sources or services 116, 118, 120, 122 and 124 shown in FIG. 1, or various Cloud services 210 in FIG. 2, may refer to and comprise any suitable database(s), database management system(s), server(s) to facilitate management, provision, transfer, and analysis of various patient healthcare information. For example, the data source/service 116 of FIG. 1 may include a database system comprising at least one hardware processor, an application platform, a network interface, tenant database for storing tenant data, system database for storing system data, program code for implementing various functions of the data source/service 116, and process space for executing database system processes and tenant-specific processes, such as running applications as part of an application hosting service.

In one aspect, the data source/service 116 may be an on-demand database service, which is made available to users outside of the enterprise(s) that own, maintain or provide access to the database system of the data source/service 116. Such users generally do not need to be concerned with building or maintaining the data source/service 116. Instead, resources provided by the data source/service 116 may be available in various contexts when the users need services provided by the data source/service 116; that is, on the demand of the users. Some on-demand database services may store information from one or more tenants into tables of a common database image to form a multi-tenant database system.

The data source/service 116 may include an application platform configured to allow various applications to execute. In some embodiments, such application platform may be configured to enable the creation, management and execution of one or more applications developed by the provider of the on-demand database service, users accessing the on-demand database service via user systems, or third party application developers accessing the on-demand database service via such user systems.

In another aspect, the data source/service 116 may be configured to implement a web-based customer relationship management (CRM) system. For example, the data source/service 116 may include application servers configured to implement and execute CRM software applications as well as provide related data, code, forms, renderable web pages and documents and other information to and from user systems and to store to, and retrieve from, a database system related data, objects, and web page content. In addition, the data source/service 116 may provide tenant access to multiple hosted (standard and custom) applications, including a CRM application. User or third party developer applications, which may or may not include CRM, may be supported by the application platform of the data source/service 116. The application platform may be configured to manage the creation and storage of the applications into one or more database objects and the execution of the applications in one or more virtual machines in the process space of the data source/service 116.

In accordance with aspects of the present disclosure, the data source/service 116 may be configured to provide web pages, forms, applications, data and media content to user (client) systems to support the access by these user systems as tenants of the data source/service 116. The data source/service 116 also provides security mechanisms to keep each tenant's data separate unless the data is shared.

Another data source/service 118 of FIG. 1 may include a marketing automation and customer engagement computing platform for supporting multi-channel campaign execution, dynamic customer journeys, pre and post campaign analytics including audience building and segmentation, social media engagement and advertising, and a data management platform.

In an example, the data source/service 118 may be configured to comprise at least one hardware processor, an application platform, a network interface, tenant database for storing tenant data, system database for storing system data, program code for implementing various functions of the data source/service 118, and process space for executing database system processes and tenant-specific processes, such as running applications as part of an application hosting service. A client's multi-channel journey enabled in the data source/service 118 may be facilitated via real-time customer engagement, email and marketing automation, social media engagement, and advertising, mobile messaging and push notifications and customer marketing analytics.

In some implementations, messages may be generated and delivered by the data source/service 118 to each contact based on an individual's current data and new messages may be triggered based on real-time customer data changes and interactions. The journeys may have multiple branches and decisions may go on different branches based on dynamically changing contact data and/or journey data. For example, data associated with a contact may indicate to where the data source/service 118 sends information. Contact data or any change in the data may determine which journey branch to take. For example, a contact may be in a communication journey for prospects but then converts to a customer. The change in the contact data in the system 100 may automatically remove the contact data from a prospect journey and place the contact data into a new customer journey. Further, journey data may indicate how a contact has interacted with a journey (e.g., email opens or clicks).

In accordance with aspects of the present disclosure, for a newly onboarding member, after an appointment has been successfully scheduled, the member may receive multiple notifications including but not limited to Email 1—welcome, Email 2—reminder to complete insurance coverage information. Email 3—recommendation for an upcoming appointment, and so on. The data source/service 118 may be configured to detect when the member is opening and interacting with each email. For example, in response to determining that Email 1 has not been opened or clicked by the member after a certain configurable time (e.g., 10 days), the data source/service 118 may be configured to send Email 2, or alternatively send Email 3 if the member has not opened and interacted with Email 2.

The data source/service 118 may also be configured to allow the management computing device/interface 114 to implement journeys using emails and short message service (SMS) or any suitable messaging methods. For example, emails may be used to communicate content related to all available services and supports provided by the system 100 of the present disclosure for a member. SMS may be used to communicate more timely information such as reminding a member to provide insurance coverage information within 24 hours prior to a scheduled appointment. Moreover, emails and SMS may be used to share post-purchase communications. For example, a journey may be created by the data source/service 118 with emails for shipment status of teeth aligners, up to the point that the aligners have been delivered, at which time the data source/service 118 may send an email/SMS message to the member to inform her the package was delivered, and then follow with a customer survey, if needed. In an embodiment, the data source/service 118 may be configured to generate personalized email content and subject lines based on an onboarding member's attributes and associated data and rules applied to the member. For example, the email content may populate based on the message recipient, thereby delivering a personalized experience without generating multiple versions of a single email. In one embodiment, data management implemented by the data source/service 118 may be performed using data extensions (e.g., a table) that may be associated to form a relational database supported with SQL.

In the context of the system 100, communications to members may originate either through a transaction (e.g., an appointment was booked) or through an automated process (e.g., a check-up reminder). The data source/service 118 may send email and SMS messages to members. For example, when a transaction triggers a communication to a member through an action on the website, the management computing device/interface 114 may issue a message that is received by the data source/service 118, which in turn sends the message to the member. A copy of the message may be sent to a Service Cloud, such that users (e.g., studio front desk and member experience agents) have a record of the communication.

Automated communications are triggered in the data source/service 118 through the creation of automated journeys that select members that should be targeted. The data source/service 118 has details about the patient appointment and treatment history from which it can be determined when a given message should be sent.

Automated communications may also be copied over to the Service Cloud. For example, a custom component “Engagement Hub” may be developed to provide users (e.g., studio front desk and member experience agents) visibility about the automated communications. The “Engagement Hub” also provides a summary of phone calls members make to the member experience team of the system 100.

In accordance with aspects of the present disclosure, the data source/service 118 may be configured to integrate with the CRM of the data source/service 116 via the management computing device/interface 114 (e.g., REST API or SOAP API), such that emails/SMS of the data source/service 118 may be sent directly from the CRM of the data source/service 116, and email engagement metrics may be tracked at the contact level in the CRM of the data source/service 116. Specific events or actions in the CRM of the data source/service 116 may act as triggers for specific journeys in the data source/service 118.

The data source/service 118 may also be configured to incorporate artificial intelligence technology and provide engagement scoring to predict who will interact with messaging, and predict the optimal time and frequency to send messages to each member of the system 100 to increase member engagement.

Another data source/service 120 of the present disclosure may include a practice management platform configured to maintain and manage various information related to members of the system 100 and healthcare or services providers (e.g., physicians, office staff, pharmacists, or labs). For example, data sources/services 120 may maintain and manage any confidential or publicly available dental and general medical information of each member of the system 100 collected from various data sources by the Cloud management server system 126. Example medical information may include, but not limited to, any information relating to a patient's dental records, health conditions, medical conditions, characterizations, assessments, test results, biographical or demographical information, prescription information, immunization records, care services provided, insurance policy information, coverage/benefits guidelines/rules for care services, healthcare plans, explanations of benefits, and the like. In one example, the data source/service 120 may be configure to assign a unique identifier for each patient, such as all information related to the patient may be organized, managed and stored by referencing to the unique identifier within a database associated with the data source/service 120.

In one aspect, the data source/service 120 may be configure to allow the dental studios of the system 100 to manage day-to-day activities including scheduling appointments, communicating with patients, documenting patient charts and compiling notes, billing patients and insurance companies, time tracking for office personnel, processing insurance claims, processing payments (including credit card processing), sharing data with other authorized providers, etc. In one preferred embodiment, the data soure/service 120 may be Cloud-based comprising at least one hardware processor, an application platform, a network interface, various databases for storing user and system data, program code for implementing various functions of the data source/service 120, and process space for executing database processes, such as running applications as part of an application hosting service.

Further, the data source/service 120 allows all provider locations of the system 100 to share data including a signal patient record, single provider record, insurance carrier list and fee schedules, coverage tables, procedure codes, and clinical notes templates. Appropriate system and function access may be assigned and granted to employees and office staff of each location of the system 100 for increased data privacy and security. The data source service 120 may be configured to track each patient's current insurance eligibility, archive expired plans, and manage multiple insurance plans per patient. For each provider location of the system 100, the office personnel may view the entire day's appointments, schedule operatories and providers, enter treatment plans and confirm appointments.

The data source/service 120 may also allow the office personnel to digitally submit and track insurance claims, optimize collections, collect accurate patient portions at time of service regardless of dental plan types, using auto-calculated coordination of benefits, accurate sliding fees, and customizable fixed copays for health maintenance organization plans (HMOs) and other fixed copay plans.

Each member of the system 100 may have a member profile (e.g., unique system identifier) managed by the data source/service 120. For example, patient dental records, account status and other important information all appear on one screen, enabling faster check-in at each location of the system 100. Online booking allows members to book re-care appointments online, at their convenience, instead of at checkout time. In addition, the status of each patient at each appointment location may be updated and displayed in the office's PC which helps to move patients efficiently through their visits.

The dental imaging processing capability included in the data source/service 120 may help all dental care providers and locations of the system 100 to store, share, and attach images to patient records and insurance claims. The patient charting software in the data source/service 120 may add procedures, conditions, and clinical notes to patient charts and group related procedures into multi-codes. Furthermore, the dental care providers may use the data source/service 120 to create dental treatment plan chart with built-in templates and descriptions, thereby improving case management by automatically tracking plan acceptance, scheduling and progress.

The data source/service 120 may be configured to provide customizable symbols shown for each tooth that can be overlaid with recent periodontal results. Current and historical images may be accessible in a dashboard and enlarged and annotated. Progress notes may be viewed and selected for editing. Clinical notes may be entered manually or through voice periodontal charting, in a resizable, movable window, while dashboard data remains in view.

Cloud management server system 126 may be scalable by adding additional data source/service 122. For example, a Cloud-based dental insurance verification and claim submission system may be configured to connect with the management computing device/interface 114. In one aspect, such a system may provide a suites of APIs that provide a universal schema to normalize data across insurance carriers. That is, members' dental eligibility benefit information obtained from various payers may be organized ina standardized and structured manner no matter each payer's information formatting or how it is conveyed to practices. As a result, dental care providers may receive the same formatted response from payers in the structured manner in which they consume data that otherwise would be an unstructured and copious mess.

Another data source/service 124 example may comprise an electronic distributed computing system for processing referral marketing sales transactions using networked computing devices of the system 100, and for enabling referral marketing communications among these computing devices. For yet another example, an artificial intelligence based diagnostic system or an expert or knowledge based medical diagnostic system may be added and obtained recommendation information therefrom may include text, audio, video, and other rich media explanations.

As will be described fully below, the system 100 of the present disclosure may connect with at least one 3^{rd} party or proprietary order management system and process various E-commerce transactions (e.g., single product purchase, subscription purchase or any applicable purchases) by e.g., creating a draft order in a shopping cart followed by a checkout process. In one aspect, the system 100 may be configured to accept a variety of payment methods through a single API and save payment details (credit card information or banking information) for future transactions by the same onboarding member.

In accordance with important aspects, the present disclosure relates to appointment scheduling based at least on dynamically changing patient preferences and dental care practitioners' availability. For example, in certain circumstances, telehealth appointments and virtual visits (e.g., video conferencing platforms offered by Zoom, MicroSoft Teams, Skype, Webex Meetings, BlueJeans Meetings, GoTo Meeting, Google Workspace, join.me) may be desired and scheduled accordingly. It should be appreciated that the present disclosure may be implemented in a variety of different contexts including but not limited to appointment scheduling for other clinical practices, consulting services, real estate agents, mechanics, architects, legal service providers, salons, or other service providers. On the provider side, the system 100 of the present disclosure may be configured to implement an availability engine to provide many options for schedule fulfillment with access to a larger consumer base, thereby significantly increasing revenue opportunities. This may be accomplished by efficiently and timely scheduling appointments, filling gaps in a schedule, and rescheduling appointments or accommodating cancelations. On the member side, the scheduling mechanism of the present disclosure advantageously allows each member of the system 100 to schedule an appointment using a single, integrated system based on a number of criteria ranging from desired dental care or treatment, geographic location, cost calculation, and user ratings of the healthcare providers. That is, the system 100 may be configured to provide a member with the flexibility and ease to select various dental treatments without having to individually search or schedule with a specialist directly.

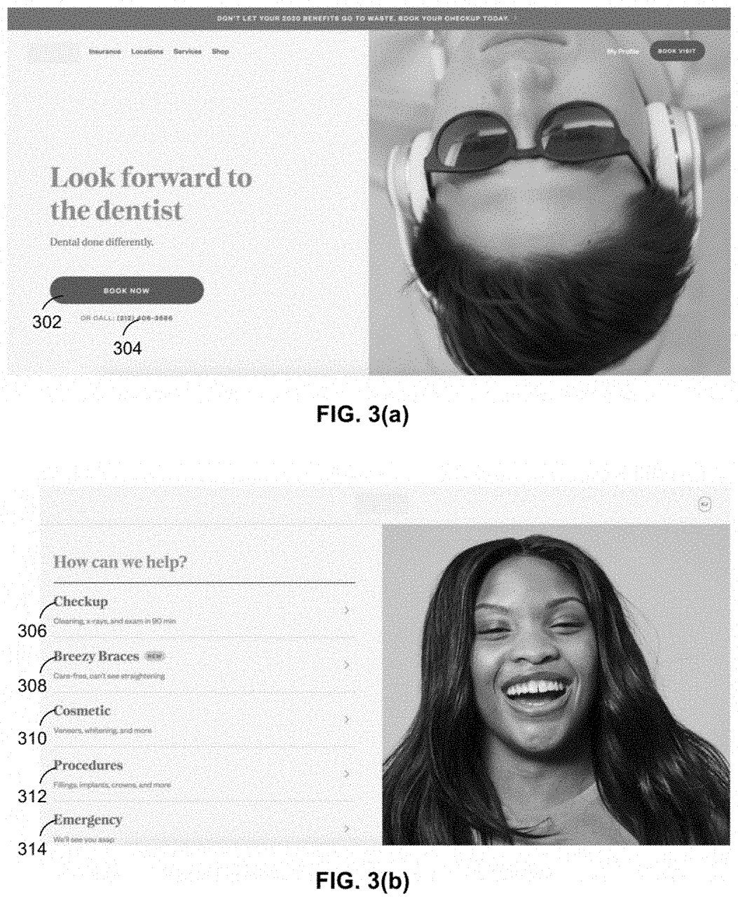

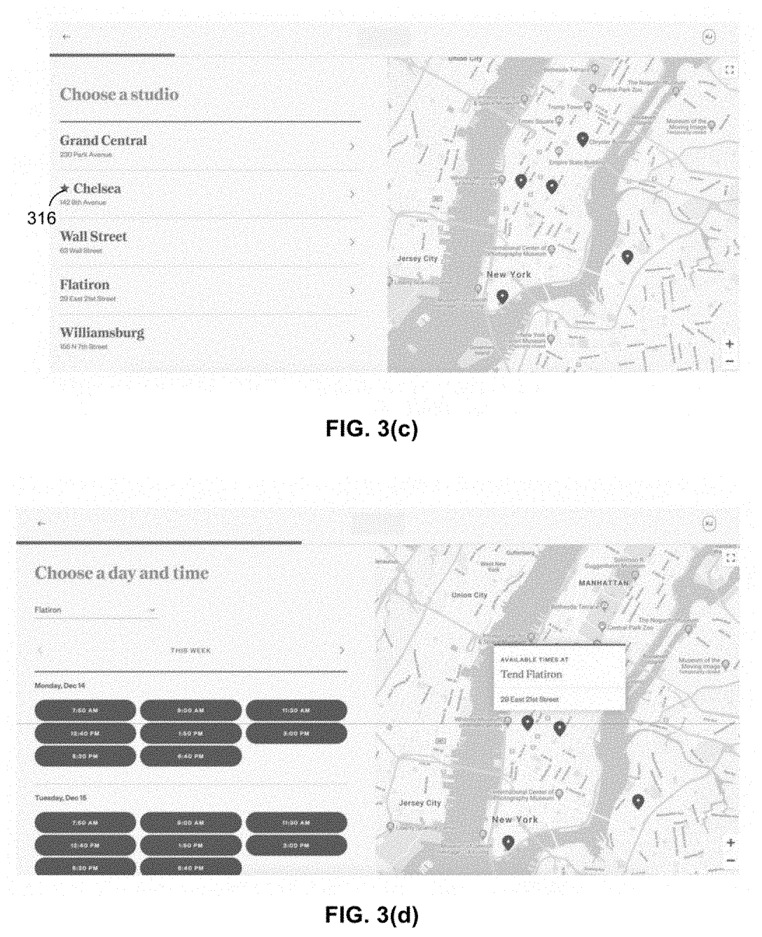

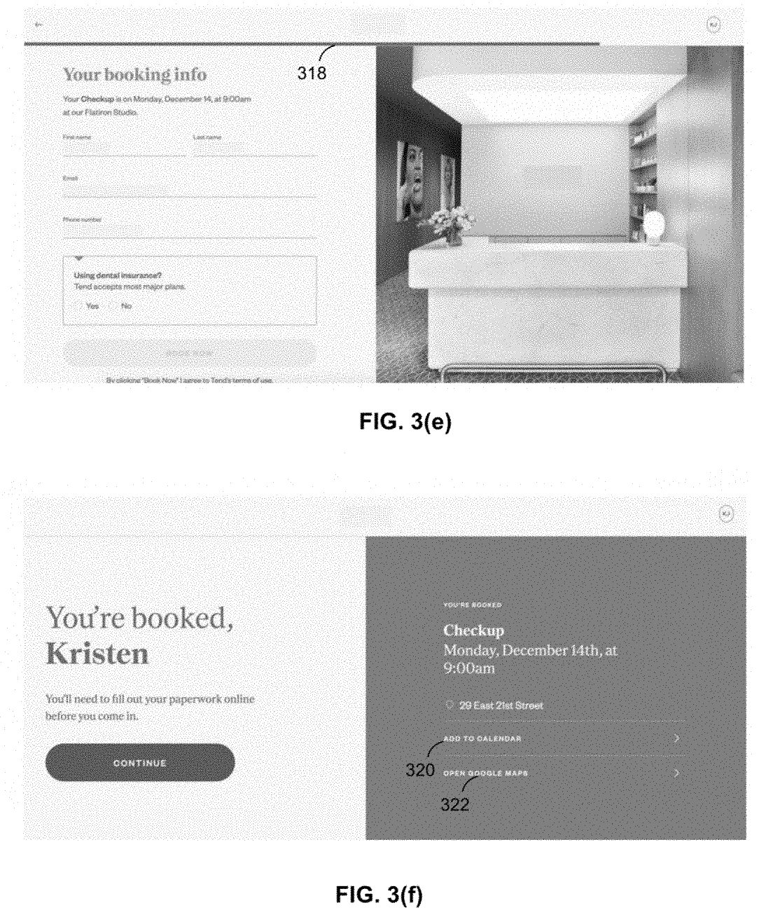

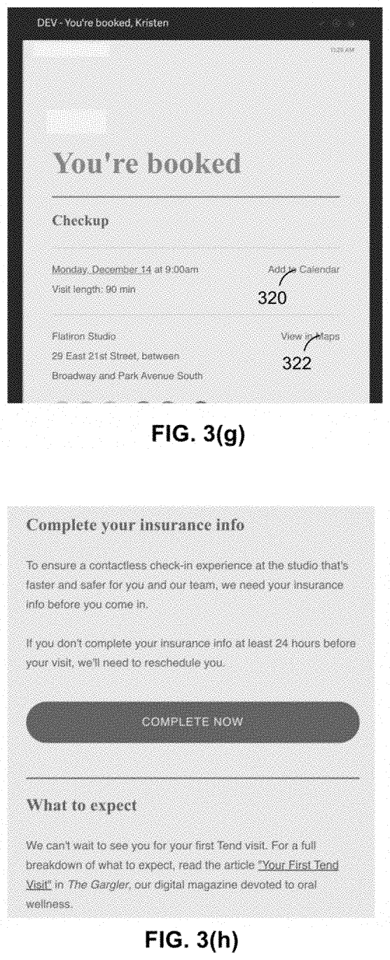

Referring to FIGS. 3(a)-3(h), the management computing device-interlace 114 of system 100 may be configured to provide an onboarding member with the ability to book a variety of different services online and generate different intake flows depending on a selected dental service. Referring to FIGS. 3(a) and 3(b), a mobile or web-based application may be instantiated via a selected computing device 104, 106 or 108 of FIG. 1 when a member connects with the system 100's customer facing website. An appointment (checkup 306 including cleaning, X-rays and oral exam, orthodontic treatment 308, cosmetic treatments 310 including veneers, whitening and more, various dental procedures 312 including fillings, implants, crowns and more, and emergency treatment/consultation 314) may be scheduled via an online process (e.g., by clicking “Book Now” button 302) or calling 304 a service number. Alternatively, the management computing device/interface 114 may be configured to allow the member to initiate a call from browser directly.

For example, when a member calls through a phone system, the number that receives the phone call is tied to e.g., Amazon Connect phone system, which in turn routes the phone call through an Interactive Voice Response (IVR) system. The IVR in Amazon Connect may be integrated with Salesforce Service Cloud through the Salesforce Service Cloud Voice application. As a result, the IVR system may be configured to look up a patient's unique identifier assigned by the system 100 based on the member's phone number and retrieve the patient's appointment history based on the system identifier of the patient.

If the IVR determines that the patient has an upcoming unconfirmed appointment, it may offer the caller the option to confirm the appointment during the call. If the patient wishes to confirm the appointment, the IVR may generate a request to an endpoint of the management computing device/interface 114 that will confirm that patient's appointment.

In another embodiment, in response to detecting that the member is selecting checkup during booking and that the member has previously visited (e.g., logging into a registered account of the system 100), the management computing device/interface 114 may be configured to route the member to a separate re-care flow and retrieve and present previously provided information. As shown in FIG. 3(c), the online user interface may display service provider locations available nearby, including a star 316 for a previously visited location by the member. As shown in FIG. 3(d), for an online booking intake, the management computing device/interface 114 of system 100 may be configured to implement a booking module/engine to display dental care provider availability for the selected service and update such information in real-time.

In one embodiment, upon receipt of the member's request for an appointment at a specific studio location, the booking module/engine may be configured to retrieve existing calendar information of the studio, prioritizing a selected period of time (e.g., next 7 days). In one embodiment, an example payload generated for the member's appointment request may include: aggregate_requests_starts_at: 2022-03-11T17:59:58.785Z; ends_at: 2022-03-16T16:59:58.785Z; patient_id: 03c7fb7e-242c-4b49-a912-6ab0519cbacc; service: CLNCHK; starts_at: 2022403-14T16:59:58.785Z; studio: rockefeller-center. The management computing device/interface 114 may be configured to generate a response with a payload including: location_id: “db081728-1250-44e3-8d55-8d6e433087f4,” service_type_id: “85afb4e3-eaee-4dce-8b0d-0cbe6de1bc20,” timeslots: . . . ends_at “2022-03-14T09:00:00-04:00,” operatory_id: “2022-03-14T09:00:00-04:00,” provider id: “a80a07e5-0504-422a-b271-22ad706e1787,” starts_at: “2022-03-14T08:00:00-04:00.” That is, the management computing device; interface 114 may be configured to make requests to the data source/service 120 of the FIG. 1 to inquire at least the following: for the given dates requested, during which hours is the studio open?; for the given service type and patient type, what operatories are available for booking?; for those operatories and during those open hours, what appointments already exist, and when?; for those operatories and during those open hours, what other “events” exist that already occupy time?. In response, the data source-service 120 may process data for each operatory including retrieving each studio's schedule, subtracting existing appointments and events, determining what time slots are available, and sending relevant information to the management computing device/interface 114.

In some implementations, appointment availability may be generated within the upcoming 2 weeks. If a member navigates forward in time (i.e., beyond the next 2 weeks), another set of requests may be generated, and another set of requests may be issued accordingly, as described above.

Retrieved calendar information may include a defined time period for all scheduled appointments, such as the start date and start time of each appointment, and optionally a duration and/or end time of the appointment. The appointment information may include a textual description of the appointment, such as information describing the purpose, topic, subject matter of the appointment. Contact information, such as the names and phone numbers associated with the studio location and parties, may also be included in the appointment information. Using location-specific scheduling rules (i.e., the availabilities are specific to operatories which are uniquely associated with a specific studio), the booking module/engine of the management computing device/interface 114 may determine available slots based on gaps in calendar equal to length of requested service. In the meantime, if the member elects to change service or appointment location, the booking module/engine may be configured to repeat the foregoing steps based on the updated member inputs and display appointment availability to the member.

In some embodiment, a given appointment timeslot is not, at present, reserved or held until the final “Book” button is clicked by a member and the management computing device/interface 114 and/or the data source/service 116 have time to process and complete such booking request. If concurrent users are online and person A completes a booking into a timeslot into which person B had just confirmed a booking (a request has been generated by clicking the “Book” button on an application), the management computing device/interface 114 may be configured to return an error message to person A who may then be redirected to start another booking process.

In accordance with aspects of the present disclosure, the booking module/engine of the management computing device/interface 114 may be configured to obtain a base set of inputs that may be used to determine to inform an availability engine. For example, one of the inputs may include a patient type (e.g., a new or existing patient). The patient type may have an effect on the type of dental procedures and the duration that may be calculated. For example, the booking module/engine may obtain the member's phone number and email address (e.g., from member inputs during the booking process or from other data sources) for checking against records saved in a database associated with the management computing device/interface 114. In response to detecting that the phone number and the email address have no match in any of the saved records, the management computing device/interface 114 may be configured to create and save a patient record in its corresponding database. In one aspect, a chart number may be uniquely generated and assigned to the new patient. Further, the system 100 may also generate and assign a unique identifier for referencing each member of the system. The database of the management computing device/interface 114 may be configured to have a bi-directional or unidirectional synchronization protocol with the database of the data sources/services 120 of FIG. 1. Record mapping relationship may be established by the unique system identifier associated with each record saved in the database of the management computing device/interface 114 and the unique patient identifier associated with each record saved in the database of the data sources/services 120. For example, the data sources/services 120 may push new additions (e.g., new patients information) and updated contents of all existing patients to the database of the management computing device/interface 114 on a configurable time period (e.g., every 5 minutes) by referencing the unique patient identifier associated with each record saved in the database of the data sources/services 120. For another example, the management computing device/interface 114 may create a new patient profile and query the data sources/services 120 to retrieve all pertinent patient information (e.g., based on the patient's demographic information and/or insurance coverage information), and manage retrieved information internally based on the unique chart number and system identifier of the particular patient. In some implementations, the synchronization protocol among different databases may use an encrypted network connection to ensure the confidentiality of the data transmitted therebetween. Moreover, the database files of the management computing device/interface 114 and the data sources/services 120 may be encrypted at the file-system level. Any applicable and appropriate data compression and conflict detection techniques may be implemented with the synchronization protocol of the present disclosure to ensure better performance and data integrity.

In another example, upon checking the member's phone number and email address (e.g., from member inputs during the booking process or from other data sources) against all records saved in the database associated with the management computing device/interface 114, the booking module/engine may determine that the member previously purchased products from the system 100 but has never booked any appointment at any dental studio. Similarly, the management computing device/interface 114 may be configured to create and save a patient record in its corresponding database and start synchronizing patient records with the data sources/services 120 via the new patient's chart number, system identifier and record identifier of the data sources/services 120.

In yet another example, upon checking the member's phone number and email address (e.g., from member inputs during the booking process or from other data sources) against all records saved in the database associated with the management computing device/interface 114, the booking module/engine may determine that the member is an existing patient of the system 100 (e.g., previously booked an appointment followed with finished or unfinished studio visit). In either case, the management computing device/interface 114 may exchange the latest information of the patient with the data sources/services 120 and update/synchronize patient specific information saved in both systems via the patient's unique chart number, system identifier and record identifier of data sources/services 120.

Further, appointment location or studio location may be key parameters for the availability engine's calculation. Another input may relate to service type. A preset list of service types that support both new and existing patients may be maintained by the management computing device-interface 114. Each service type may be catered toward a specific service provided by the system 100 and the management computing device/interface 114 is set up to book for. It should be appreciated that a wide variety of service types of the system 100 that inform the availability engine may be expanded upon dynamically. When a member opts for an appointment slot during an online booking process, the service dates may be passed along in such a request to the data source/service 120 in order to base data needed to calculate availability. In one aspect, all availability calculation may be configured to begin by fetching appointments and event blocks from the data source/service 120's API. Event blocks may include spans of time that represent blocked availability.

In an embodiment, the availability engine may be configured to initially select a service type, service dates, and appointment location. Each service type provided by the system 100 may beset up to be scheduled into a specific suite depending on if the patient is new or existing. Here, a suite is synonymous with operatory, which may refer to where a patient physically sits during a dental procedure at the selected appointment location. A service type configuration for a basic hygiene visit may look like the following: new patients will be booked into Suite A while existing patients get booked into Suite B; and new patients will be scheduled for 60 minutes, whereas an existing patient will be scheduled for 50 minutes.

Next, the availability engine may be configured to request appointment, event block, and studio data from the data source/service 120 where such information has been maintained and managed. After all the required information is gathered, the availability engine may be configured to generate a series of requests to the data source/service 120 in order to get a base set of data to make calculations upon. In some embodiments, the management computing device/interface 114's API may request the following data from the data source/service 120 using request service date as boundaries: all currently active appointments, event blocks (events that block availability) and studio hours (business hours the studio operates within).

In accordance with aspects of the present disclosure, the availability engine may be configured to calculate real-time availability via a timespan inversion algorithm. Once all requests are made, for a given studio, the management computing device/interface 114's API may begin calculation by iterating through each suite's all currently active appointments and event blocks to determine what times are not available. Next, the API may invert each timespan based on whatever hours remain.

As an example calculating process, Suite A has up to 8 hours available hours per day; for a given date, Suite A has 4 hours of active appointments and 2 hours of event blocks (i.e., time for which an operatory is not available for clinical use); the aforementioned timespan inversion algorithm may commence by merging all appointments and event block timespans together. Once merged, the management computing device/interface 114's API may determine that there are 6 hours of blocks time and then invert the 6-hour block of time to identify what time remains. In this instance, 2 hours remains.

Once a set of real-time available timespans have been fully calculated for a given suite, the management computing device/interface 114's API may be configured to apply service type booking rules based on the patient type and service type inputs. As an example, the management computing device/interface 114 may be configured to apply a set of business rules to affect availability outputs as the following: a member requests availability for basic cleaning, which spans 60 minutes for new patients and 30 minutes for existing patients; the management computing device/interface 114's API inversion algorithm may be configured to determine that there are 2 hours available for Suite A; the outputs may be dependent on the patient type: for new patients, there are only two availability timeslots; for existing patients, there are four available timeslots. That is, the management computing device/interface 114's API may segment each available time span by service duration followed by serializing requisite booking information per time slot. Thereafter, serialized availability of the dental studio maybe returned and presented to the member for confirmation. Once confirmed, the management computing device/interface 114 may be configured to validate requisite booking information to ensure that the appointment exists, the appointment is associated with a specific member, and the appointment date and time is not within any block availability etc. In addition, the management computing device/interface 114 may be configured to publish the appointment information to the data source/service 120.

Referring to FIG. 3(e), if the member has previously visited a provider studio and has logged in into her member account, certain booking information (e.g., name, email address, and phone number) may be automatically retrieved and displayed by the booking module/engine of the management computing device/interface 114. In one embodiment, a progress tracker 318 across the top of the user interface may indicate how many steps are remaining until finishing the online booking flow. Upon receiving the member's confirmation of a selected appointment, as shown in FIGS. 3(f) and 3(g), the booking module/engine of the management computing device/interface 114 may be configured to synchronize 320 the calendar information with a local calendar application of the member's computing device and display public transportation 322 which is dynamic based on the appointment location. As illustrated in FIG. 3(h), the booking module/engine may also be configured to prompt the member to complete additional information in advance of a visit, present pre-visit content detailing what to expect from the visit, and send email confirmations. In some embodiments, if the member fails to complete the insurance information at least 24 hours before the scheduled visit, the booking module/engine may be configured to generate and transmit a reminder to the member and/or reschedule the visit. Notification changes dynamically depending on nature of service.

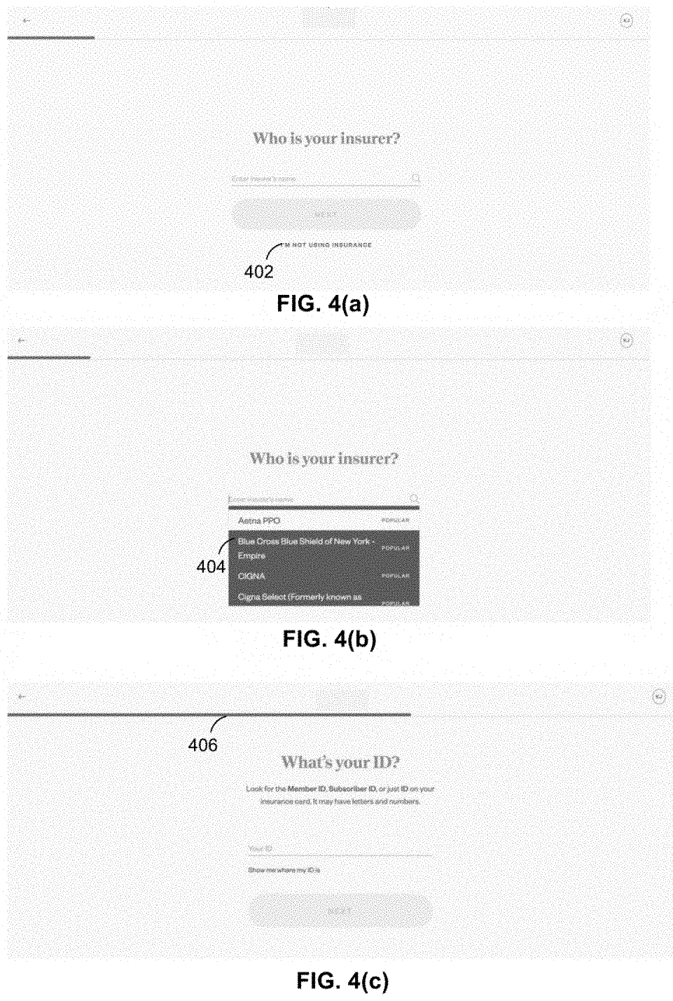

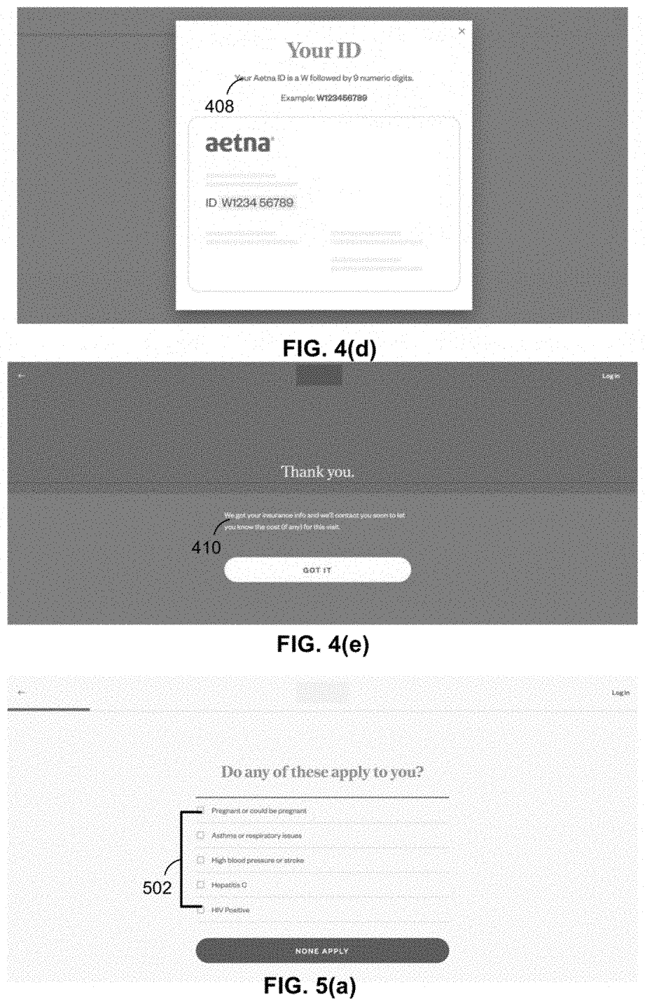

In accordance with aspects of the present disclosure, the management computing device/interface 114 of system 100 may be configured to obtain and manage member insurance coverage information. If an onboarding member plans to use insurance for an upcoming visit, a series of screens and prompts may be configured to guide the member to enter insurance information, as shown in FIGS. 4(a)-4(e). If no insurance is used 402, the management computing device/interface 114 may be configured to determine cash pay pricing, such that the member knows the cost for the upcoming visit. As shown in FIG. 4(b), a drop down list of insurers 404 may be displayed, sorted by the most commonly selected insurers. Further, a progress tracker 406 across the top of the user interface may indicate how many steps are remaining until fully entering the insurance coverage information. In one embodiment, based on the insurance payer selected by the member, the management computing device/interface 114 may dynamically determine and generate message and example 408 to help the member locate and enter a correct insurance identification number, as shown in FIG. 4(d). Upon obtaining the insurance information, the system 100 may be configured to calculate and send the cost for the selected service type to the member prior to the scheduled visit, as shown in 410 of FIG. 4(e). Moreover, the management computing device/interface 114 may automatically input relevant information into a dental insurance verification and calculation system (e.g., the system 122 in FIG. 1), such that when a treatment is recommended, the system 100 may accurately estimate the cost of additional services. In some embodiments, the member receives a patient-facing high level check and a provider-facing detailed check. The provider-facing detailed check may be helpful for doing detailed treatment plans in a timely fashion while the patient is in chair.

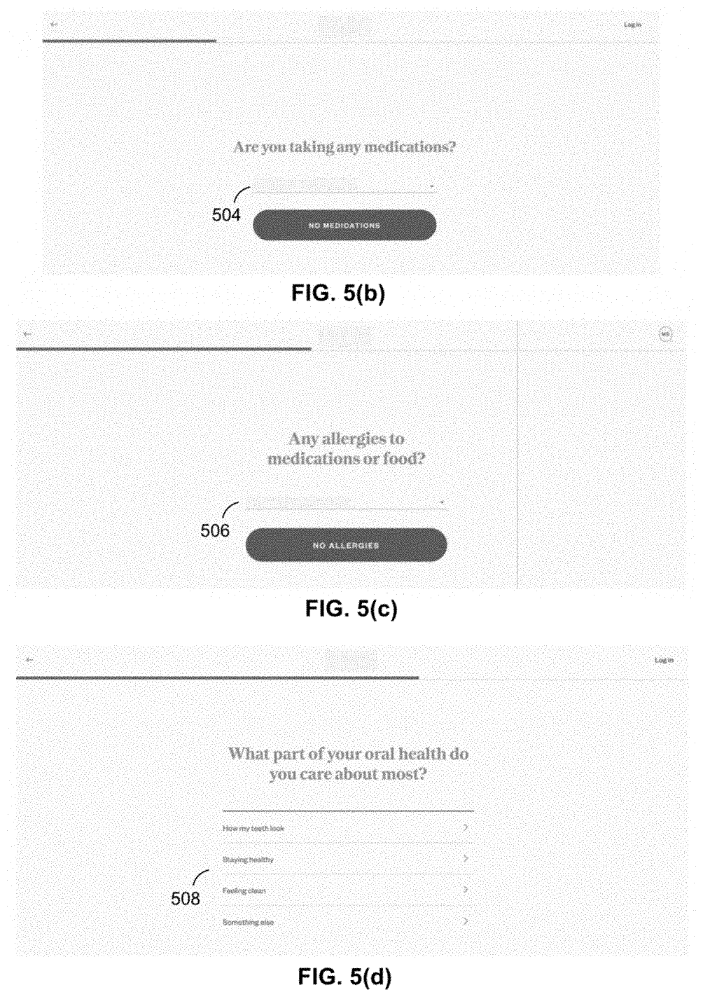

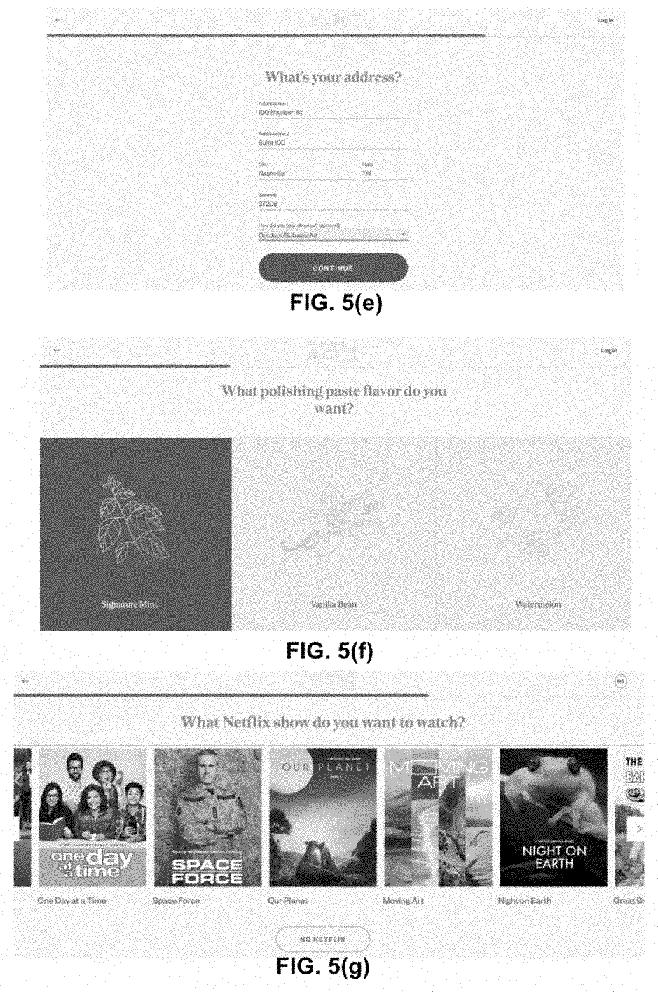

Now referring to FIGS. 5(a)-5(g), the system 100 of the present disclosure allows an onboarding member to provide information to personalize an upcoming visit and treatment experience. For example, as shown in FIG. 5(a), a plurality of screening questions may be determined and presented to a pre-appointment member. Example questions 502 may include pregnancy, asthma or respiratory issues, high blood pressure or stroke, Hepatitis C and HIV/AIDS information. As respectively shown in FIGS. 5(b)-5(d), the member may be prompted to enter prescription information 504, allergy information 506 or expectation for personal oral health 508. Member inputs to these screening questions may be automatically transmitted to the management computing device/interface 114 and other pertinent servers/systems (e.g., the data source/service 122 of FIG. 1) for storage, further processing and/or analyzing, and recommending how a provider treats the member during the scheduled visit. In one example, if a member indicates that she is pregnant, X-rays may not be performed during her visit. Further, if a member is on anticoagulants, she may be advised to speak with the dental care provider if an oral surgery is to be scheduled. If a member is allergic to antibiotics, an alternative such as Clindamycin may be prescribed to reduce the risk of infection. If a member answers the question shown in FIG. 5(d) “What part of your oral health do you care about most?” with “How my teeth look,” a dental care provider may offer to provide more information about whitening, orthodontics, or veneers in studio. Alternatively, if a member answers the question with “Staying healthy,” a dentist may have a conversation with the patient regarding the aspects of her oral health that matter the most, and accordingly tailor recommended treatment.

FIGS. 5(e)-5(g) show additional personal information gathered pre-appointment for facilitating a personalized clinical experience of an upcoming visit. For example, the member may be prompted to enter a current residential address (FIG. 5(e)), favorite polishing tooth paste flavor (FIG. 5(f)), and preferred shows to watch during the scheduled visit (FIG. 5(g)). The management computing device/interface 114 may transmit member inputs to, e.g., the data source/service 120 for storage, such that studio staff can tailor the clinical experience based on member preferences. For example, staff ensures that the member's favorite paste flavor is used during the scheduled visit and the preferred show is pre-loaded and ready to watch prior to the member's visit.

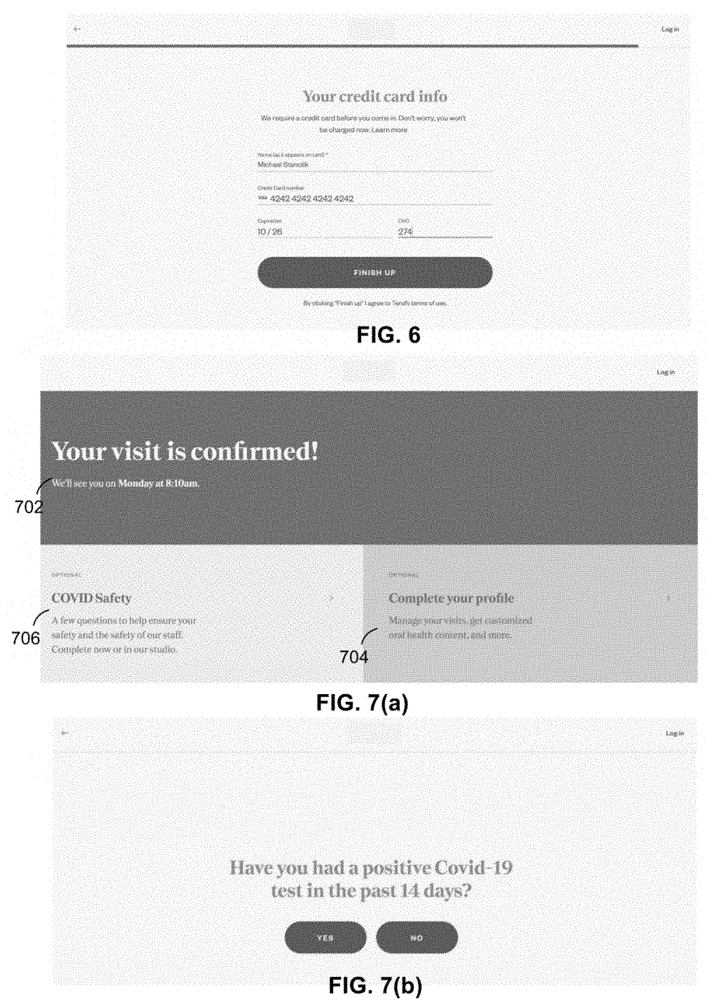

Payment information may be collected at the end of a booking process. By providing credit card information in advance of a visit, a member may leave at the end of the visit without having to provide any additional payment and/or go through a checkout process. In one embodiment, as shown in FIG. 6, the management computing device/interface 114 may be configured to connect with a payment processing system to obtain credit/debit or banking information (e.g., card/account number, expiration date and card verification code (CVC)) from an onboarding member. If the member does not wish to provide payment information during booking, member experience team can collect over the phone or in studio staff can collect in person via a custom digital point of sale (POS) device. The payment processing system may be configured to review and verify obtained financial information. In one aspect, account tokens and/or person tokens may be generated to ensure all personally identifiable information are secure. The management computing device/interface 114 may be configured to save these generated tokens that reference the customers, thereby ensuring the system 100 is payment card industry (PC) compliant. When a member completes a visit, staff may complete payment via a custom digital POS device without requiring additional credit/debit card information. Such payment request may be transmitted to the payment processing system for processing with a specific credit card vendor.

In an embodiment, the system 100 of the present disclosure may implement and configure a POS module which is a custom component that was developed to allow users (studio staff and member experience agents) to make credit card charges against member credit cards.

Member credit card payment methods may be stored in an online payment processing platform for Internet businesses. For example, all of the patient credit card information may be maintained and managed in a platform account and each studio location has its own sub-account such that charges by the location can be identified. When a charge is made, customer payment information may be cloned from the platform account and used in the studio sub-account. The transaction details are also recorded in a custom service and a patient's transaction history may be displayed across multiple studio locations. The presentation of credit card transactions on the patient page also enables refunds on a specific transaction, if necessary. Daily transaction reporting by studio location may be implemented to enable studio end-of-day charge reconciliation as part of daily closing activities.

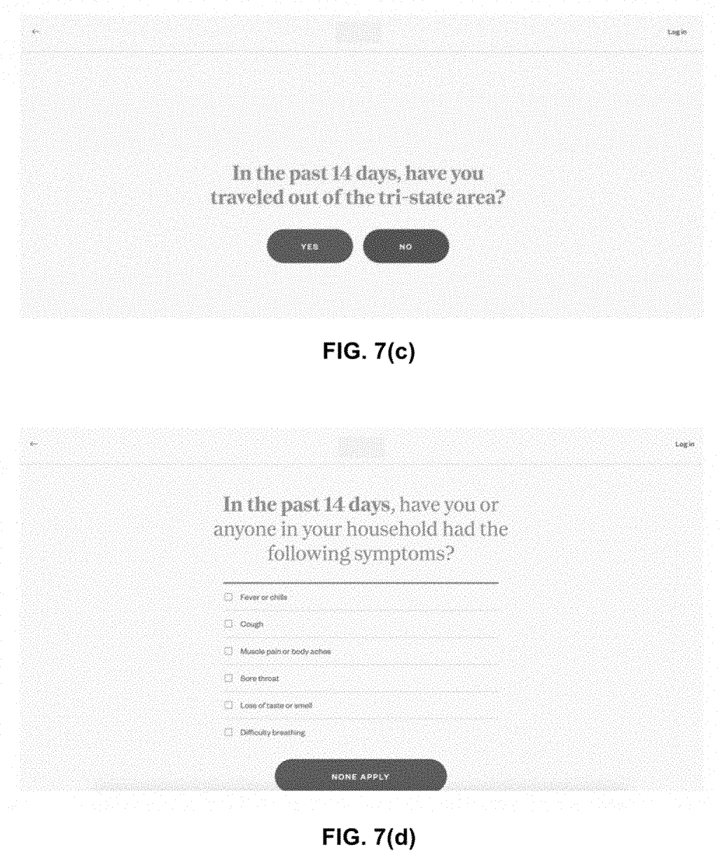

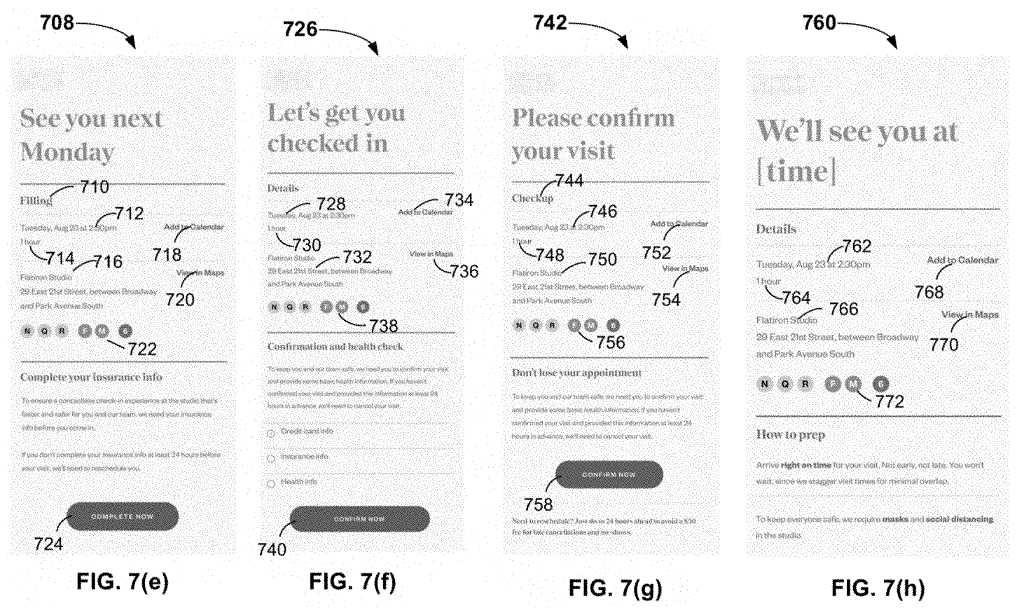

One of the main elements of patient-centered care is an enhancement of patient preparedness. Referring now to FIGS. 7(a) 7(h), after an online booking process has been completed, the system 100 of the present disclosure may be configured to determine and generate a variety of pre-visit planning assessment questionnaire and notifications to prepare and involve patients in their treatment processes. For example, as shown in FIG. 7(a), in addition to a brief confirmation message 702 of an upcoming visit, the management computing device/interface 114 may be configured to generate and display multiple optional messages 704 to an onboarding member based at least upon whether the member has provided certain information during the booking process. In one embodiment, a COVID-19 related prescreening scrip 706 before the member's arrival may help healthcare providers manage the safe reopening of their practices and emphasize new precautions that must be taken to protect patients, clinicians and staff from COVID-19 as in-person care resumes or becomes more routine. It should be appreciated that the management computing device/interface 114 of the present disclosure may be configured to generate any suitable prescreening script in connection with any regional and global situation and contingency. That is, the system 100 of the present disclosure may be configured to effect tele-triage of patients in order to keep practice staff and visitors safe while putting patients on the appropriate care path as some who seek an in-person visit may be better accommodated through a telehealth virtual visit or, in more urgent cases, need to be directed to a hospital or COVID-19 testing site. As shown in FIGS. 7(b) 7(d), the management computing device/interface 114 may be configured to present a pre-visit screening questionnaire to an onboarding member prior to her visit as a part of an effort to reduce community spread of COVID-19. Further, reaching out to patients to explain safety procedures in advance of their visit can help to alleviate patient anxiety by assuring them that the practice has protocols in place.

A variety of notifications, as shown in FIGS. 7(e)-7(h), may be dynamically generated and transmitted to a member based at least upon whether the member has completed certain booking information or confirmed the upcoming visit. For example, as shown in FIG. 7(e), if a member has scheduled a visit without insurance coverage information on file, the management computing device/interface 114 of the present disclosure may be configured to generate and transmit a notification 708 to the member prior to the visit. In one embodiment, such notification 708 may indicate appointment type (fillings) 710, appointment date and time 712, appointment duration 714, and appointment location 716. The booking module/engine of the management computing device/interface 114 may be configured to synchronize 718 the calendar information with a local calendar application of the member's computing device, and display map information 720 and public transportation information 722 based on the appointment location. In one aspect, notification 708 reminds and invites 724 the member to provide her insurance coverage information at least 24 hours before the scheduled visit or an appointment rescheduling may be arranged.

Referring to FIG. 7(f), the management computing device/interface 114 of the present disclosure may be configured to generate and transmit a check-in notification 726 to a member prior to a visit. In one embodiment, such notification 726 may indicate appointment date and time 728, appointment duration 730, and appointment location 732. The booking module/engine of the management computing device/interface 114 may be configured to synchronize 734 the calendar information with a local calendar application of the member's computing device, and display map information 736 and public transportation information 738 based on the appointment location. In one aspect, notification 726 reminds and invites 740 the member to provide her credit card information, insurance coverage information and health information at least 24 hours before the scheduled visit or an appointment rescheduling may be arranged.

FIG. 7(g) illustrates an example visit confirmation notification 742 to a member prior to a visit. Such notification 742 may indicate appointment type (check-up) 744, appointment date and time 746, appointment duration 748, and appointment location 750. The booking module/engine of the management computing device/interface 114 may be configured to synchronize 752 the calendar information with a local calendar application of the member's computing device, and display map information 754 and public transportation information 756 based on the appointment location. In one aspect, notification 742 reminds and invites 758 the member to confirm at least 24 hours before the scheduled visit or an appointment rescheduling may be arranged.

In response to detecting that the member has confirmed a visit, a notification 760 may be generated and displayed by the management computing device/interface 114 to the member. Such notification 760 may indicate appointment date and time 762, appointment duration 764, and appointment location 766. The booking module/engine of the management computing device/interface 114 may be configured to synchronize 768 the calendar information with a local calendar application of the member's computing device, and display map information 770 and public transportation information 772 based on the appointment location. In one aspect, notification 760 may include essential recommendations, suggestions, and precautions for the office visit.

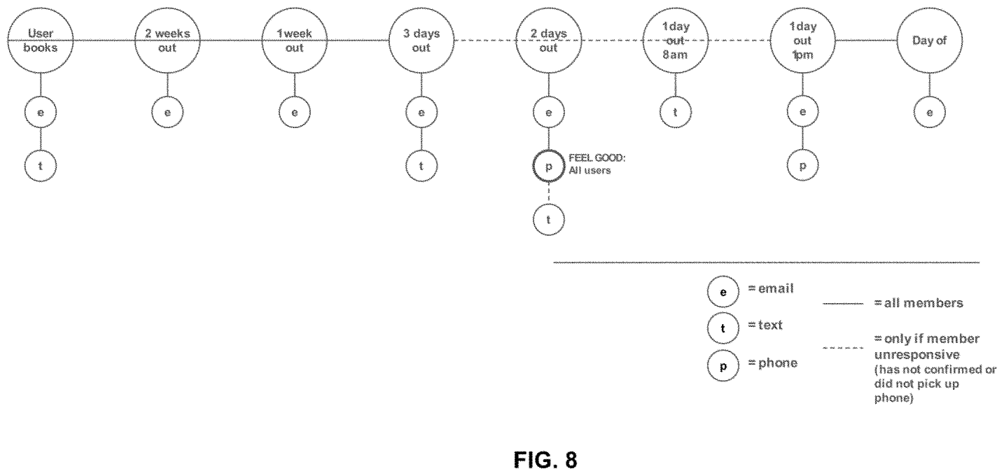

FIG. 8 shows a process of automated messaging workflows based on obtained member data relating to an upcoming visit. In an aspect, the management computing device/interface 114 of the present disclosure may be configured to generate and transmit a series of notifications (e.g., emails, text messages, and phone calls) to the member leading up to a visit based on predetermined intervals (e.g., two weeks, one week, 3 days, 2 days, 1 day and on the day of the visit).

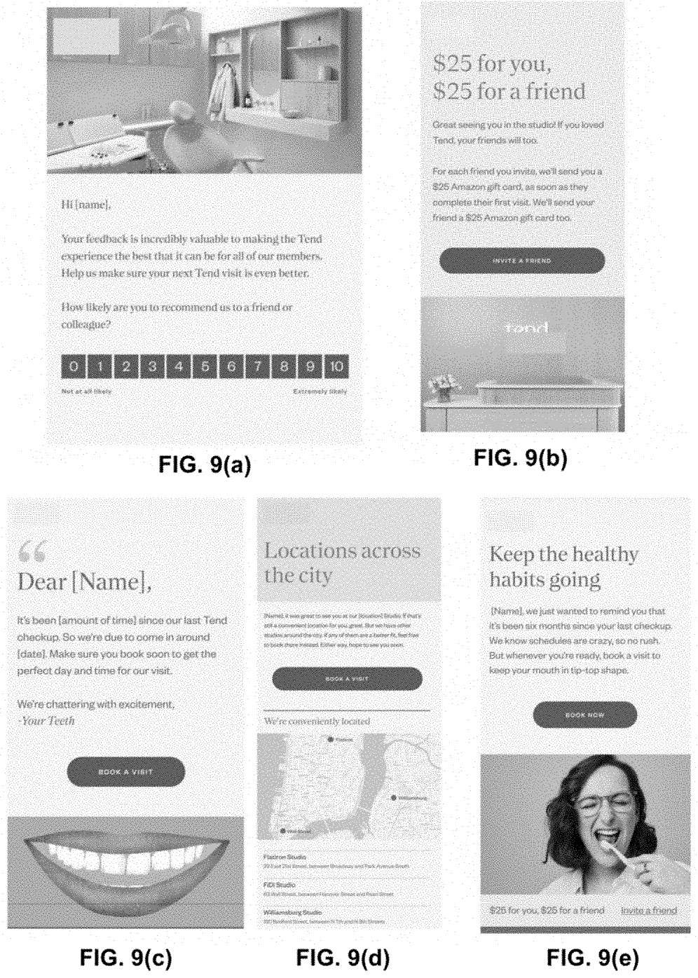

FIGS. 9(a)-9(e) illustrate a variety of post-visit notifications to a member including but not limited to feedback/recommendation (FIG. 9(a)), referral campaigns (FIG. 9(b)), re-care reminder for follow up hygiene visits after the initial visit (FIGS. 9(c) and 9(e)), recommendation for additional appointment locations (FIG. 9(d)).

In one aspect, the management computing device/interface 114 may be configured to solicit a member that has completed an appointment at one of the dental studios for a review of her experience via one of several online review aggregation platforms (e.g., Google and Yelp). In one embodiment, such feedback solicitation process may be automated using SMS or any suitable messaging methods.

As disclosed above, all appointments at all dental studios associated with the system 100 may be maintained and managed in the data source/service 120 shown in FIG. 1. When an appointment is completed, that appointment may be assigned an indicator “complete” within the data source/service 120 and its associated computing devices. In the meantime, the data source/service 120 may be configured to expose a REST API that allows authenticated member computing devices (e.g., computing devices 104, 106, 108 of FIG. 1) to obtain the present state of certain data (e.g., appointment information).

The management computing device/interface 114 of the present disclosure may leverage this capability by querying the data source/service 120 for the latest information about scheduled appointments according to a defined schedule which may be configured within a computing platform deployed with the Cloud management server system 126 (e.g., AWS's “CloudWatch” platform). In one embodiment, such a computing platform may be configured to emit one or more events to the management computing device/interface 114 of the present disclosure according to the defined schedule. These events, when received by the management computing device/interface 114, may prompt it to request appointment data from the data source/service 120 via e.g. REST API. The internal syntax/structure/protocols used by these AWS CloudWatch events may be abstracted from the system 100, as consumers of the CloudWatch service.

Thereafter, the management computing device/interface 114 may be configured to store the receive appointment data within a database (e.g., PostgreSQL) which may be hosted in AWS's relational database service (RDS) platform. In one aspect, the appointment data may be configured to reflect any new appointments that have been created since the previous scheduled invocation, as well as any changes to existing appointments. These changes may include a status update for an appointment. For example, if the status of an appointment has changed from anything (“Booked”, “Late”, etc.) to “Complete”, the management computing device/interface 114 may determine that this appointment is transitioning to a completed status.

When the management computing device/interface 114 handles an appointment that is transitioning to “Complete” status, it instantiates a process that may potentially result in the member being solicited to leave a review for received services.

First, the management computing device/interface 114 may be configured to query its own databases (e.g., PostgreSQL, AWS RDS) to determine if a record of such a solicitation being sent already exists. This is to ensure that appointments that may be re-transitioning to “Complete” do not produce additional, excess review solicitations. That is, such database queries handle edge cases wherein members may receive excess solicitations.

If no record of an earlier solicitation exists, the management computing device/interface 114 may configured to issue a solicitation. For example, it may begin by determining which appointment location or studio the appointment occurred in. The management computing device/interface 114 may check data from its PostgreSQL database and find a percentage value specific to that studio (e.g., a percentage of solicitations desired for marketing purposes). The number may be, for example, 60%. This number will be referred to as the studio's solicitation split. The management computing device/interface 114 may be configured to produce a random number between 1 and 100. If the number is above the solicitation split, review solicitations are handled via a third-party computing platform (e.g., Podium). If the number is below the solicitation split, review solicitations are handled via another third-party computing platform (e.g., the data source/service 118 of FIG. 1).

In one embodiment, if the review solicitations are handled by Podium, the management computing device/interface 114 may generate an HTTP request to Podium's REST API—specifically a POST request to produce a “switchboard_invitation” entity. The HTTP request payload may include the member's (i.e., who was present for the completed appointment) phone number, email address, first name, last name, and the identifier of the studio in which the appointment occurred. In accordance with predetermined configuration between the management computing device/interface 114 and Podium's web application user interface (UI), creation of these Switchboard Invitation entities may result in an SMS message being sent to the member that directs her to leave a review in e.g., Google or Yelp. In one aspect, the content of the text message may be configurable by the system 100 via Podium's web application UI.

If the review solicitations are handled by the data source/service 118, the management computing device/interface 114 may be configured to generate an HTTP request to the data source/service 118's REST API—specifically a POST request that includes the member's “Person Contact ID” (an internal ID for Salesforce, corresponding to the member within that system), the identifier of the studio in which the appointment occurred, the member's phone number, the member's “person ID” (an internal system identifier corresponding to the member within the system 100), and the SMS keyword of “SMILE.” That data is received by the data source-service 118 and, per the API key contained within the request's target URL, the data received may be composed with an SMS template within the data source/service 118's system that the system 100 produced and maintains. This results in a complete, personalized SMS message directing the specific member to Yelp to leave a review for the received dental services.

Thereafter, the management computing device/interface 114 awaits the results of either call (to Podium or to the data source/service 118). Upon receiving an HTTP response code indicating success of the API call, the management computing device/interface 114 may be configured to generate a record of a solicitation and store it within its PostgreSQL database. As described earlier, this prevents repeated subsequent solicitation issuances for the same appointment. In one embodiment, this record may include a link to the appointment for which the solicitation was issued, the “kind” of solicitation (Podium or Yelp), and the external ID of the original solicitation as it exists in either system.

If the management computing device interface 114 receives an HTTP response code indicating a failure, a log record may be created describing the failure, including the original response from the API (Podium or the data source/service 118).

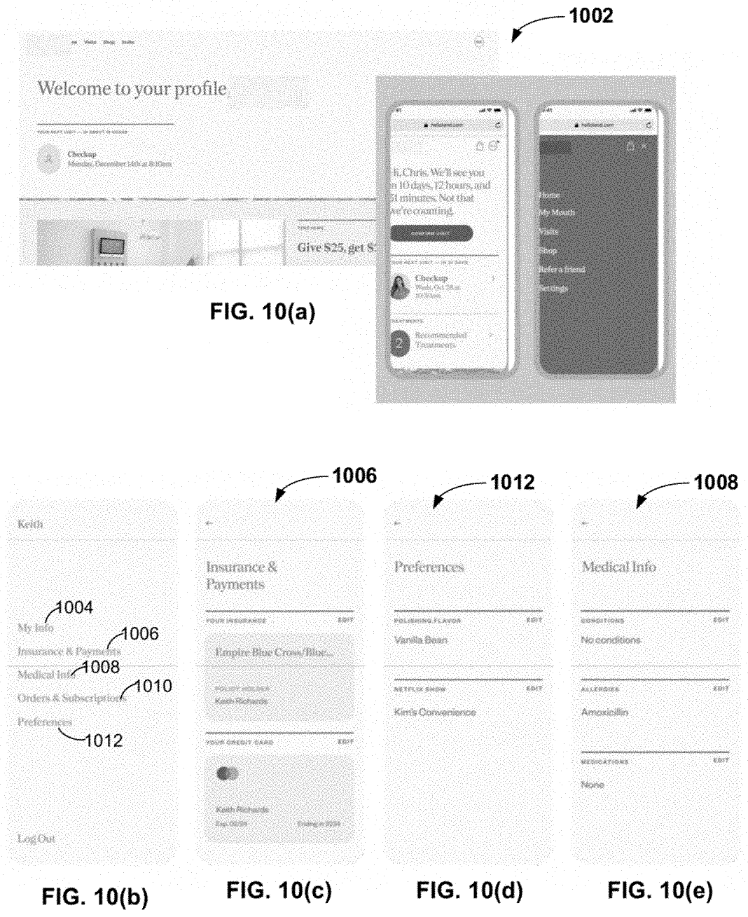

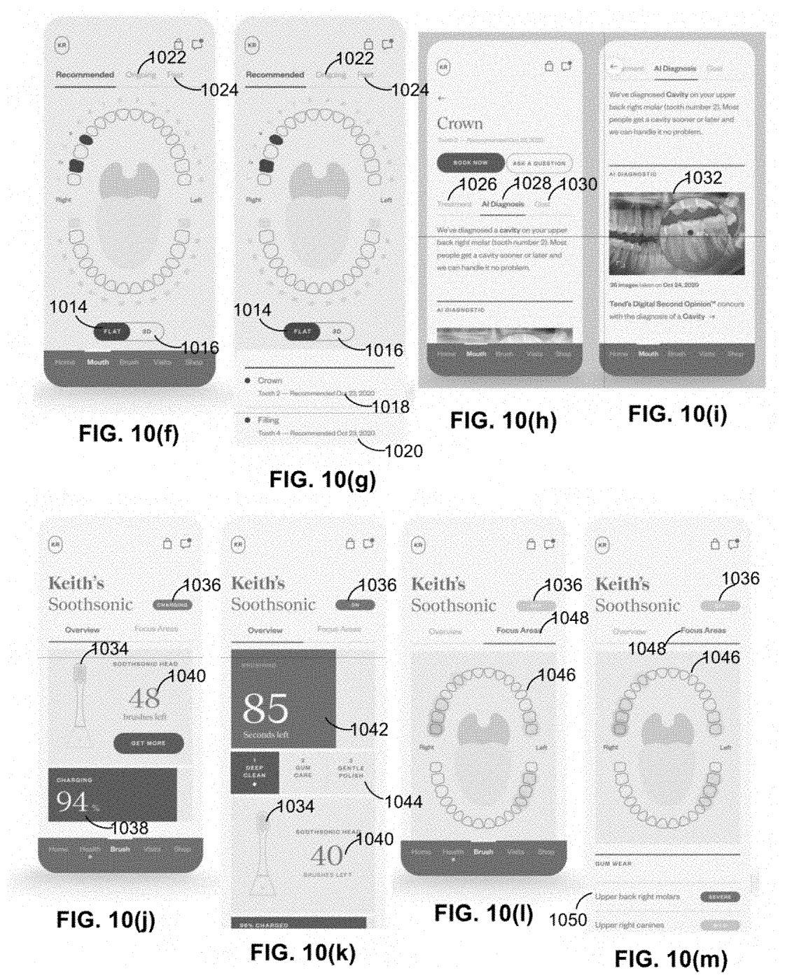

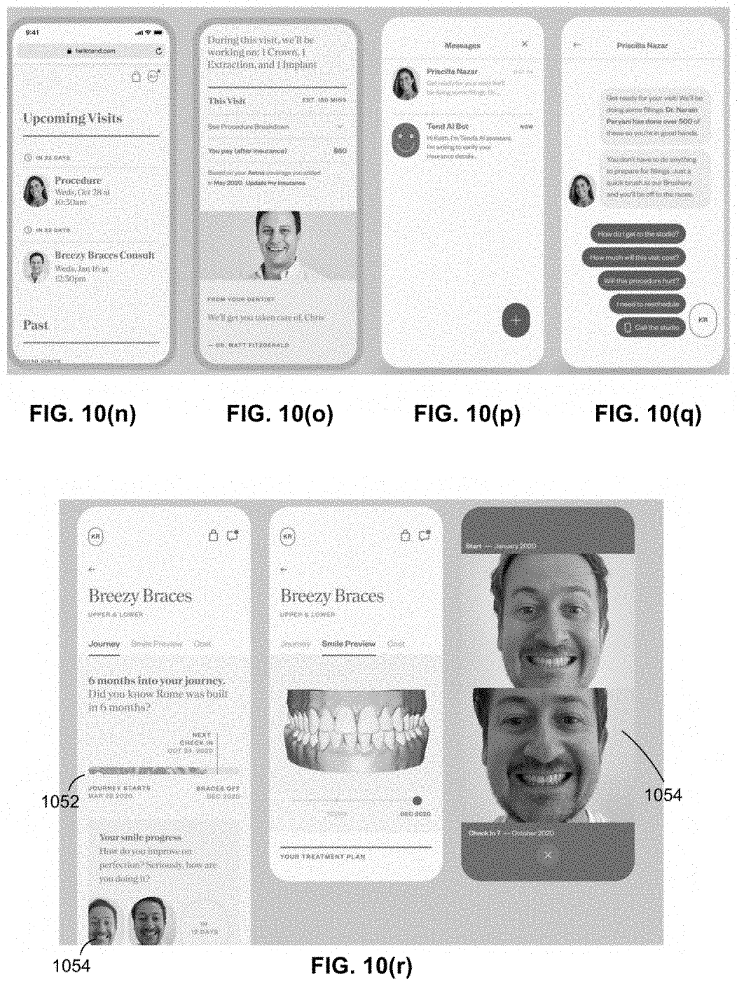

In accordance with important aspects of the present disclosure, referring back to FIG. 1, a user (e.g., at least one of user 102a, 102b . . . 102n) may install or instantiate a mobile or web-based application (e.g., native iOS or Android apps) via a selected computing device 104, 106 or 108. Here, an application may refer to a computer program or software application designed to run on a computing device. As shown in FIG. 10 (a), an application 1002 of the present disclosure may have a user interface and home screen configured to present a plurality of elements and options to a member for managing his or her profile, exploring recommended, ongoing, and past treatments and procedures, learning about procedures with pre- and post-visit instructions that may include the member's images and dental X-rays, viewing treatment options including cost breakdowns and finance options, and scheduling treatment(s) and ask the care team questions.

In some embodiments, as shown in FIG. 10(b), an authenticated and registered member of the system 100 may log into her account via the application 1002 to manage her personal information 1004, insurance and payment information 1006 (FIG. 10(c)), medical information 1008 including medical conditions, allergies and current prescription (FIG. 10(e)), order and subscriptions 1010, and preferences 1012 including favorite tooth paste flavor and TV shows to watch during a visit (FIG. 10(d)).

As shown in FIGS. 10(f)-10(i), the system 100 may also be configured to provide treatment planning and allow the member to review each treatment plan recommended by a dentist and validated by a patient-facing Al via the application 1002. That is, the system 100 may present to the member information via the application 1002 that can be understood intuitively and provide a deep dive on the details of what each treatment is and how it works, why the targeted member would do it, how much the member should expect to pay given the insurance data provide by the member, and the ability to schedule one or more appointments directly online. Via the application 1002, the member may follow along on her journey with upcoming visits and recommended content, view progress via pictures, and have access to a dedicated care team via HIPAA secure chat, text and phone calls.

In some embodiments, the application 1002 may also allow a member of the system 100 to explore the health of her gums and areas of focus, provide personalized brush tips and product recommendations, provide visualized brush information via a connected toothbrush, and provide other engaging contents and challenges to improve her overall oral health. For example, as shown in FIGS. 10(f) and 10(g), a 2-dimensional teeth numbers chart 1014 or 3-dimensional teeth image 1016 may be presented to a member showing recommended care for one or more teeth based on the examination results obtain from the member's prior visit(s). Intuitively, all 32 teeth (molars, premolars, incisors and canines) are identified by their numbers and arranged in a clockwise fashion as seen by a dentist. Teeth numbers 1-16 are on the upper jaw. Teeth numbers 17-32 are in the lower jaw. As an example, teeth numbers 1, 16, 17, and 32 indicate wisdom teeth which are a type of molars. Teeth numbers 14 and 15 represent upper left molars. If a member is interested in getting cosmetic dentistry using veneers, a dentist may enhance the most visible part, teeth numbers 6-11 on the upper jaw and 22-26 on the lower jaw. Member's prior visit(s) may identify area of focus such as tooth decay and cavities. For example, recommended treatments 1018 (crown) and 1020 (filling) for teeth No. 2 and No. 4 respectively may be presented to the member with a recommendation date. The application 1002 may also allow the member to review ongoing and past treatments and procedures 1022 and 1024. As shown in FIG. 10(h), if the member is interested in the recommended treatment for tooth No. 2, the application 1002 may organize and present detailed treatment information 1026, Al diagnosis 1028 and cost information 1030. FIG. 10(i) illustrates how AI diagnosis provides interpretation of dental images 1032 (radiographs (X-rays), cone-bean computed tomography system images, or magnetic resonance imaging scans) obtained from member's prior visit(s), and provide recommendations and future dental disease predictions. In one embodiment, the diagnosis may assign a % probability of diagnosis for common conditions like cavities and gum disease (e.g., 92% shown in FIG. 10(i)), which is presented to the member to aide her decision. These recommendations and diagnosis may help the member treat cavities early which significantly reduces the cost of treatment, time of restoration, and risk of tooth loss. The application 1002 advantageously utilizes AI diagnostic algorithms to detect dental decay and periodontal disease, oral cancer, dental caries and provide AI-assisted orthodontic treatment planning.

In another embodiment, as shown in FIGS. 10(j) and 10(k), the application 1002 may be configured to provide visualized brush information via a smart cleaning device (e.g., an electronic rechargeable toothbrush 1034 provided by the system 100 and used by a member). For example, toothbrush 1034 may be configured to have a number of electronic components including but not limited to a power source, one or more electronic sensors, movement capture sensors, and a transmitter, such as a Bluetooth transmitter, Wi-Fi transmitter, or near field communication transmitters, for paring with the application 1002. Operational status 1036 (e.g., charging, ON, OFF) and charging percentage indicator 1038 of the smart cleaning device 1034 and recommended oral cleaning products 1040 (e.g., replaceable toothbrush heads) may be displayed to the member via the application 1002. As the member is brushing her teeth using the toothbrush 1034, the operational status 1036 of the toothbrush 1034 is updated accordingly and a countdown timer 1042 may be configured to display the remaining brushing time for a member selected cleaning mode 1044 (e.g., deep clean, gum care or gentle polish).

In one aspect, various sensors associated with the smart cleaning device 1034 may be configured to determine breath freshness, degree of tooth staining, bacteria count on the teeth and gums, and/or gum health based on detected signals as the member is brushing her teeth. Specifically, these sensors may be configured to detect the position of the smart cleaning device 1034 relative to features of the oral cavity, and the location and amount of bacteria may be mapped to a model 1046 of the oral cavity. Information about the location, amount, or other characteristics of the bacteria may then be transmitted from the smart cleaning device 1034, for display to the member on the application 1002 (e.g., Focus Areas 1048 in FIGS. 10(l) and 10(m)). In some embodiments, as shown in FIG. 10(m), detailed explanation and severity or criticality of the identified dental issues 1050 may be organized and presented to the member.

Referring now to FIGS. 10(n)-10(q), the application 1002 may also allow a member of the system 100 to book and manage upcoming visits, view itemized cost breakdowns for visits including payment options and pre-approvals, access her care team via chat, text and phone, and provide tele-visits and tele-consults.

In accordance with aspects of the present disclosure, the system 100 of the present disclosure may be configured to guide an interested member to book a consult appointment via the application 1002 and create an entity in a database coupled to the management computing device/interface 114. Here, a database entity may refer to a thing, person, place, unit, object or any item about which the data should be captured and stored in the form of properties, workflow and tables. While workflow and tables may be optional for a database entity, properties are generally required for defining an entity. Candidate status, patient responsibility, insurance coverage, and promotional amount may be added via the CRM of the data source/service 116 of FIG. 1. These data may be submitted to the management computing device/interface 114 and populate the generated database entity for the specific member. If the member is not a candidate for the orthodontic treatment provided by the system 100 after all obtained data have been reviewed, the process ends.

Alternatively, if the member is determined to be a candidate, an assigned dental code D8090 may be created in the database as part of an orthodontic treatment plan. This code is commonly used by dental insurers for adults who are undergoing occlusion and alignment corrections. When the D8090 code is “COMPLETED” in the data source/service 118 of FIG. 1, the management computing device/interface 114 updates the Breezy Braces Entity status to “ACCEPTED.”

In one aspect, via the API of the management computing device/interface 114, the status of the member's scans, wires, and wire shipment may be ingested and displayed via e.g., Breezy Braces treatment experience in the application 1002. In the meantime, the creation of a bonding appointment in the data source/service 118 triggers. The creation of a bonding appointment determines if the member is “in treatment.” As shown in FIG. 10(r), treatment duration 1052 and progress photos 1054 may be added by the CRM to the application 1002 during treatment and continue to populate the Breezy Braces database entity. In the data source/service 118, when a patient has a bonding appointment, and de-bonding appointment that is COMPLETE within the timeframe of the beginning and end of the Breezy Braces database entity, the management computing device/interface 114 determines the journey to be completed.

In one embodiment, the system 100 of the present disclosure may be configured to provision push notifications between the application 1002 running on a member's computing device (e.g., devices 104, 106, 108 of FIG. 1) and one or more servers corresponding to the application 1002.

In one embodiment, push notifications may be initiated through an Email Activity that is placed in journey in the data source/service 118 that controls the business logic for a communication. The Email Activity references an email template that uses server side Javascript (SSJS) to compose a payload and send an API request to a proprietary application configured to abstract common communication tasks instead of sending a traditional email. In one embodiment, such a proprietary application may be configured to accept declarative requests (e.g., “send an SMS to person A”, “send a push notification to person A”. “send an email to person B”) from other computing devices of the system 100 and carry out the requested communication tasks.

For registration and validation, the member's computing device (e.g., devices 104, 106, 108 of FIG. 1), whether an iOS or Android platform, may be prompted for permission which results in a messaging service (APNS/FCP) token if granted. Device token may be sent to an endpoint in the proprietary application which in turn sends a request to Amazon SNS based on the platform type (iOS/Android) which registers the device. For example, device information (token) may be sent to the proprietary application such that it may establish push notification functionality for that device, and it stores the device data in its own database such that subsequent push notifications may be sent to that device/member. Device identifier may be stored in a database in correspondence with a person identifier of the system 100 that is included in the original request. When the device is registered, an Amazon Resource Name (ARN) may be returned to the device as a token and saved in storage.

In accordance with aspects of the present disclosure, the management computing device/interface 114 may be configured to perform insurance verification and defect detection.

In one embodiment, certain valid U.S. based insurance demographic data may be required for processing insurance verification: member identifier assigned by the insurance carrier, group number belonging to the insurance carrier, the insurance carrier that demographic data originates from, first and last name of the policyholder or dependent to receive medical procedures, and policyholder first and last name of the insurance holder.

Further, another parameter for insurance verification may include current dental terminology (CDT) codes which are a set of medical codes for dental procedures that cover oral health and dentistry. Each procedural code is an alphanumeric code beginning with the letter “D” (the procedure code) and followed by four numbers (the nomenclature). It also includes written descriptions for some of the procedural codes. For each insurance plan, coverage dates describe the date range for which an insurance plan is active. In addition, coverage exceptions may occur during insurance verification to downgrade certain CDT codes to a different CDT code. For instance, a tooth porcelain crown may be downgraded to a different type of crown.

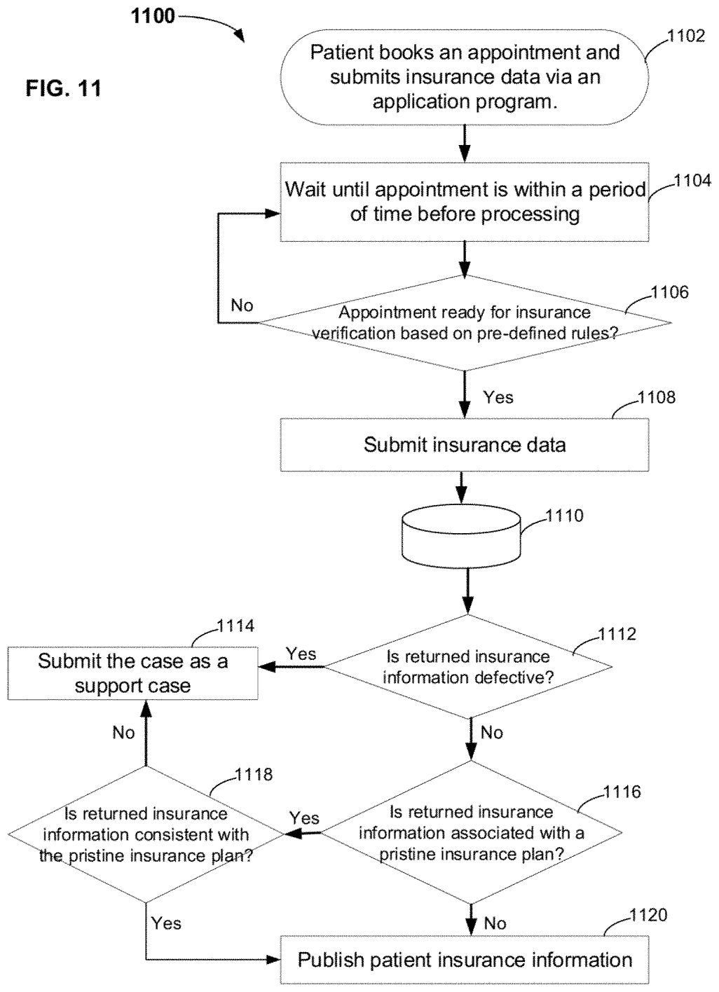

In one aspect, the management computing device/interface 114 may be configured to initiate an insurance verification process 1100 for a member upon obtaining one or more entry points. For example, referring to FIG. 11, when an appointment is booked (1102) with the management computing device/interface 114 via the application program 1002 of FIG. 10(a) and insurance coverage information may be available via member inputs. Subsequently, the management computing device/interface 114 may be configured to wait (1104) until the appointment is within a set amount of time before processing and determine (1106) whether the appointment is ready for insurance verification based on a plurality of pre-defined rules. In an alternative embodiment, insurance demographic data may be obtained via other data sources (e.g., the data source/service 116 or 118 of FIG. 1) and submitted to the management computing device/interface 114.

Next, the management computing device/interface 114 may submit (1106) the demographic data of a member to a 3^{rd} party or proprietary database system 1110 (e.g., the data source/service 122 of FIG. 1) which may be configured to retrieve benefits data specific to the member by communicating with the insurance carrier identified by the member directly. In one embodiment, the data source/service 122 handles the normalization of these data and return them to the management computing device/interface 114 in a standardized format. At a high level, the benefits output from the database system 1110 may include policyholder maximum and deductible data, benefits broken down by CDT code, which includes age limitations, frequencies, historical data, and benefit percentages, and insurance plan coverage dates and coverage exceptions.

Thereafter, the management computing device/interface 114 may be configured to run a defect validation algorithm (1112) against the benefits output based at least on multiple pre-defined set of rules. It should be appreciated that there are a wide variety of defect validation rules in the management computing device/interface 114. As an example, for a given set of demographic data, the returned benefits information by the data source/service 122 may be partitioned by CDT codes. One of those CDT codes may be for “Periodic Evaluation.” and the actual dental code may be D0120. The management computing device/interface 114 may be configured to determine whether the insurance carrier return historical data for this CDT code. If the carrier does return history, defect detection may check if historical data is present for this CDT code. If not, the “Periodic Evaluation” procedure may be flagged as a historical defect. If the carrier does not return history, no defect will be flagged.

The management computing device/interface 114 may be configured to determine whether there is a defined age limitation. If a CDT code has an age limitation, defect detection may check if the age range is defined. If not, this CDT code may be flagged as an age limitation defect. If there is no age limitation, no defect may be flagged.

Further, the management computing device/interface 114 may be configured to determine whether there is a defined frequency. If a CDT code has a defined frequency, defect detection may check if data is present for the quantity, time period value, and time period qualifier (months, years, days). If no frequency is defined, no defect may be flagged.

In another example, the management computing device/interface 114 may be configured to determine whether benefit percentages are defined. Benefit percentages are required for all CDT codes. If one is not defined, a defect may be flagged as a defect.

There are many potential defect outputs configured in the management computing device/interface 114. Below is a partial list of defect outputs and their meanings:

| Defect Output | Definition |
|---|---|
| Plan: Missing downgrade | The insurance plan is missing benefits |
| on crowns | for downgrading on crowns. |
| Plan: Missing effective | The insurance plan is missing information |
| date | for when the plan is effective. |
| Plan: Missing group | The insurance plan is missing |
| number | a group number. |
| D0210 - Missing time | CDT code D0210 is missing |
| period qualifier | its time period qualifier. |
| D0210 - Missing | CDT code D0210 is missing |
| history | historical data. |
| D0210 - Missing | CDT code D0210 is missing |
| benefit % | benefit percentage. |

If a set of demographic data is found to be defective, the result may be published to t e data source/service 116 or 118 of FIG. 1, such that a defection correction process may be carried out (1114). For example, corrections may be submitted directly to the data source/service 122. As a feedback loop, the data source/service 122 may automatically submit corrections or other inputs to the management computing device/interface 114 as they may be operated upon by team members. Then, the management computing device/interface 114 API may re-run the newly updated benefit information against the defect validation algorithm in the previous step.

If no defects have been detected, the management computing device/interface 114 API may be configured to further detect whether the returned insurance coverage information specific to the member is associated with (1116) a pristine insurance plan and consistent with (1118) the pristine insurance plan. Here, a pristine insurance plan of the present disclosure may contain information applicable to the same insurance policy number and/or the same group number. For example, in response to detect that the returned insurance coverage information share the same policy/group number of a saved pristine insurance plan, the management computing device/interface 114 may proceed to check whether the returned insurance coverage options are consistent with that of the pristine insurance plan. If the returned insurance information is not associated with a pristine insurance plan or consistent with a saved pristine insurance plan, no actions may be taken except publishing (1120) or updating insurance details for the specific member in the data source/service 120 of FIG. 1.

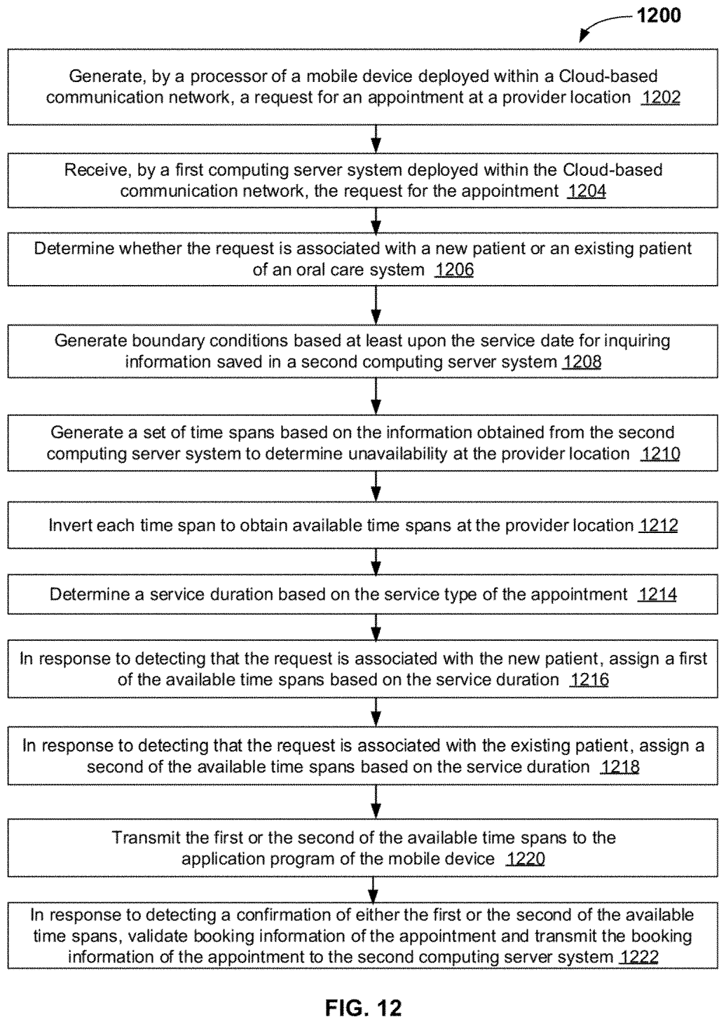

FIG. 12 illustrates a flow chart of a computer-implemented method 1200, in accordance with aspects of the present disclosure. In one embodiment, the method 1200 may comprise generating (1202), by a processor of a mobile device (e.g., member computing devices 104, 106 and 108 of FIG. 1) deployed within a Cloud-based communication network, a request for an appointment at a provider location. For example, the request may comprise data representing a phone number, an email address, a service type of the appointment, the provider location, and a service date and time. The method 1200 may further comprise receiving (1204) the request by a first computing server system (e.g., the management computing device/interface 114) deployed within the Cloud-based communication network; determining (1206), by the first computing server system, whether the request is associated with a new patient or an existing patient of an oral care system; and generating (1208), by the first computing server system, boundary conditions based at least upon the service date for inquiring information saved in a second computing server system (e.g., data source/service 120 of FIG. 1) relating to active appointments of the provider location, blocked availability and operation hours of the provider location.

The method 1200 of the present disclosure may additionally comprise generating (1210), by the first computing server system, a set of time spans based on the information obtained from the second computing server system to determine unavailability at the provider location; inverting (1212), by the first computing server system, each of the set of time spans to obtain available time spans at the provider location; determining (1214), by a first computing server system, a service duration based on the service type of the appointment. In response to detecting that the request is associated with the new patient, the method 1200 may comprise assigning (1216), by the first computing server system, a first of the available time spans based on the service duration. In response to detecting that the request is associated with the existing patient, the method 1200 may comprise assigning (1218), by a first computing server system, a second of the available time spans based on the service duration.

Moreover, the method 1200 may include transmitting (1220), by the first computing server system, the first or the second of the available time spans to the application program of the mobile device; and in response to detecting a confirmation of either the first or the second of the available time spans via the application program of the mobile device, validating (1222), by a first computing server system, booking information of the appointment and transmitting the booking information of the appointment to the second computing server system.

The above description of the disclosure is provided to enable a person skilled in the art to make or use the disclosure. Various modifications to the disclosure will be readily apparent to those skilled in the art, and the common principles defined herein may be applied to other variations without departing from the spirit or scope of the disclosure.

Furthermore, although elements of the described aspects and/or embodiments may be described or claimed in the singular, the plural is contemplated unless limitation to the singular is explicitly stated. Additionally, all or a portion of any aspect and/or embodiment may be utilized with all or a portion of any other aspect and/or embodiment, unless stated otherwise. Thus, the disclosure is not to be limited to the examples and designs described herein but is to be accorded the widest scope consistent with the principles and novel features disclosed herein.

1. A system deployed within a Cloud-based communication network, the system comprising:
a mobile device, comprising:
a non-transitory computer-readable storage medium configured to store an application program; and
a processor coupled to the non-transitory computer-readable storage medium and configured to control a plurality of modules to execute instructions of the application program for generating a request for an appointment at a provider location, wherein the request comprises data representing a phone number, an email address, a service type of the appointment, the provider location, and a service date and time; and
a first computing server system configured to:
receive the request,
determine whether the request is associated with a new patient or an existing patient of the system,
generate boundary conditions based at least upon the service date for inquiring information saved in a second computing server system relating to active appointments of the provider location, blocked availability and operation hours of the provider location,
generate a set of time spans based on the information obtained from the second computing server system to determine unavailability at the provider location,
invert each of the set of time spans to obtain available time spans at the provider location,
determine a service duration based on the service type of the appointment and whether the request is associated with the new patient or the existing patient of the system,
in response to detecting that the request is associated with the new patient, assign a first of the available time spans based on the service duration,
in response to detecting that the request is associated with the existing patient, assign a second of the available time spans based on the service duration,
transmit the first or the second of the available time spans to the application program of the mobile device, and
in response to detecting a confirmation of either the first or the second of the available time spans via the application program of the mobile device, validate booking information of the appointment and transmit the booking information of the appointment to the second computing server system.

2. The system of claim 1, wherein the first computing server system is configured to determine whether the request is associated with the new patient or the existing patient of the system by at least checking the phone number and the email address against records saved in a database associated with the first computing server system.

3. The system of claim 2, wherein, in response to detecting that the phone number and the email address have no match in the records saved in the database associated with the first computing server system, the first computing server system is configured to create a patient record, wherein the patient record comprises a chart number uniquely assigned to the new patient.

4. The system of claim 3, wherein the first computing server system is configured to assign a first identifier to each member of the system and the second computing server system is configured to assign a second identifier to each patient that has a record saved in the second computing server system, wherein the first and second computing server systems are configured to synchronize patient records in accordance with a selected time interval using the chart number, the first identifier, and the second identifier.

5. The system of claim 1, wherein the first computing server system is further configured to retrieve patient data from the second computing server system and present at least a portion of the patient data via the application program of the mobile device in response to determining that the request is associated with the existing patient of the system.

6. The system of claim 1, wherein the first computing server system is further configured to concurrently receive another request from another mobile device for the appointment at the provider location, determine which request has been confirmed first, and generate a message to redirect a later-confirmed request to another booking process.

7. The system of claim 1, wherein the first computing server system is further configured to determine any of the available time spans is at least equal to the service duration.

8. The system of claim 1, wherein the first computing server system is further configured to synchronize at least a portion of the booking information with a local calendar and map application program of the mobile device.

9. The system of claim 1, wherein the first computing server system is further configured to generate and transmit one or more notifications to the application program of the mobile device prior to the appointment.

10. The system of claim 9, wherein the one or more notifications are configured to prompt inputs of at least insurance coverage information, health information, and financial information from a patient.

11. The system of claim 10, wherein the first computing server system is further configured to reschedule the appointment for the patient if the insurance coverage information, health information, or financial information is not received within a selected period of time prior to the appointment.

12. A computer-implemented method, comprising:
generating, by a processor of a mobile device deployed within a Cloud-based communication network, a request for an appointment at a provider location, wherein the request comprises data representing a phone number, an email address, a service type of the appointment, the provider location, and a service date and time;
receiving, by a first computing server system deployed within the Cloud-based communication network, the request;
determining, by the first computing server system, whether the request is associated with a new patient or an existing patient of an oral care system;
generating, by the first computing server system, boundary conditions based at least upon the service date for inquiring information saved in a second computing server system relating to active appointments of the provider location, blocked availability and operation hours of the provider location;
generating, by the first computing server system, a set of time spans based on the information obtained from the second computing server system to determine unavailability at the provider location;
inverting, by the first computing server system, each of the set of time spans to obtain available time spans at the provider location;
determining, by the first computing server system, a service duration based on the service type of the appointment;
in response to detecting that the request is associated with the new patient, assigning, by the first computing server system, a first of the available time spans based on the service duration;
in response to detecting that the request is associated with the existing patient, assigning, by the first computing server system, a second of the available time spans based on the service duration;
transmitting, by the first computing server system, the first or the second of the available time spans to the application program of the mobile device; and
in response to detecting a confirmation of either the first or the second of the available time spans via the application program of the mobile device, validating, by the first computing server system, booking information of the appointment and transmitting the booking information of the appointment to the second computing server system.

13. The method of claim 12, further comprising determining, by the first computing server system, whether the request is associated with the new patient or the existing patient by at least checking the phone number and the email address against records saved in a database associated with the first computing server system.

14. The method of claim 13, further comprising in response to detecting that the phone number and the email address have no match in the records saved in the database associated with the first computing server system, creating, by the first computing server system, a patient record, wherein the patient record comprises a chart number uniquely assigned to the new patient.

15. The method of claim 13, further comprising:
assigning, by the first computing server system, a first identifier to each member of the system;
assigning, by the second computing server system, a second identifier to each patient; and
synchronizing patient records saved in the first and second computing server systems in accordance with a selected time interval using the chart number, the first identifier and the second identifier.

16. The method of claim 12, further comprising:
concurrently receiving, by the first computing server system, another request from another mobile device for the appointment at the provider location;
determining which request has been confirmed first; and
generating a message to redirect a later-confirmed request to another booking process.

17. A non-transitory computer readable medium storing computer executable instructions for a system deployed in a Cloud-based communication network, the instructions being configured for:
generating, by a processor of a mobile device deployed within the Cloud-based communication network, a request for an appointment at a provider location, wherein the request comprises data representing a service type of the appointment, the provider location, and a service date and time;
receiving, by a first computing server system deployed within the Cloud-based communication network, the request;
determining, by the first computing server system, whether the request is associated with a new patient or an existing patient of an oral care system;
generating, by the first computing server system, boundary conditions based at least upon the service date for inquiring information saved in a second computing server system relating to active appointments of the provider location, blocked availability and operation hours of the provider location;
generating, by the first computing server system, a set of time spans based on the information obtained from the second computing server system to determine unavailability at the provider location;
inverting, by the first computing server system, each of the set of time spans to obtain available time spans at the provider location;
determining, by the first computing server system, a service duration based on the service type of the appointment;
in response to detecting that the request is associated with the new patient, assigning, by the first computing server system, a first of the available time spans based on the service duration;
in response to detecting that the request is associated with the existing patient, assigning, by the first computing server system, a second of the available time spans based on the service duration;
transmitting, by the first computing server system, the first or the second of the available time spans to the application program of the mobile device; and
in response to detecting a confirmation of either the first or the second of the available time spans via the application program of the mobile device, validating, by the first computing server system, booking information of the appointment and transmitting the booking information of the appointment to the second computing server system.

18. The non-transitory computer readable medium of claim 17, further comprising instructions for determining, by the first computing server system, whether the request is associated with the new patient or the existing patient by at least checking the phone number and the email address against records saved in a database associated with the first computing server system.

19. The non-transitory computer readable medium of claim 17, further comprising instructions for, in response to detecting that the phone number and the email address have no match in the records saved in the database associated with the first computing server system, creating, by the first computing server system, a patient record wherein the patient record comprises a chart number uniquely assigned to the new patient.

20. The non-transitory computer readable medium of claim 17, further comprising instructions for:
assigning, by the first computing server system, a first identifier to each member of the system;
assigning, by the second computing server system, a second identifier to each patient; and
synchronizing patient records saved in the first and second computing server systems in accordance with a selected time interval using the chart number, the first identifier, and second identifier.
